(19) United States
(12) Patent Application Publication
(10) Pub. No.: US 2024/0294878 A1
(43) Pub. Date: Sep. 5, 2024

(54) METHOD OF PRODUCING EXOSOMES FROM IMMORTALIZED CLONAL MESENCHYMAL STEM CELLS (HMSCS) DERIVED FROM HLA HOMOZYGOUS HUMAN INDUCED PLURIPOTENT STEM CELLS (HIPSC'S) DERIVED FROM CORD BLOOD

(71) Applicants: Arne JENSEN, Bochum (DE); Metin COLPAN, Essen (DE)

(72) Inventors: Arne JENSEN, Bochum (DE); Metin COLPAN, Essen (DE)

(21) Appl. No.: 18/578,527

(22) PCT Filed: Jul. 13, 2022
(86) PCT No.: PCT/EP2022/069609

§ 371 Date: Jan. 11, 2024

(30) Foreign Application Priority Data
Jul. 13, 2021 (EP) 21185299.1

(51) Int. Cl. C12N 5/0775

(52) U.S. Cl. CPC C12N 5/0665; C12N 2501/115; C12N 2501/125; C12N 2501/145; C12N 2501/2306; C12N 2501/26; C12N 2502/1323; C12N 2533/52

(57) ABSTRACT

The invention relates to a method of producing mesenchymal stem cell derived exosomes comprising the steps of,

performing a method of producing a clonal mesenchymal stem cell line capable of producing exosomes (MSC-derived extracellular vesicles) comprising the steps of,

providing human induced pluripotent stem cells (hiPSCs),

generating therefrom an immortalized clonal cell line of mesenchymal stem cells (IMSCs),

characterizing the potential of the IMSCs to produce exosomes,

cultivating the desired IMSCs such that the IMSCs produce exosomes,

isolating the exosomes.

The invention relates to extracellular vesicles or exosomes, produced according to the invention.

The invention relates to the use of these EVs as a medicament and in a pharmaceutical composition.

### CROSS-REFERENCE TO RELATED APPLICATIONS

This application is a National Phase application of PCT Application number PCT/EP2022/069609 filed on Jul. 13, 2022 titled “METHOD OF PRODUCING EXOSOMES FROM IMMORTALIZED CLONAL MESENCHYMAL STEM CELLS (HMSCS) DERIVED FROM HLA HOMOZYGOUS HUMAN INDUCED PLURIPOTENT STEM CELLS (HIPSC'S) DERIVED FROM CORD BLOOD”, the entirety of the disclosure of which is hereby incorporated by this reference.

### BACKGROUND

Stem cells are undifferentiated cells that have the capacity to differentiate into two or more cell types with self-renewal capacity. Based on differentiation potency, stem cells can be classified into totipotent stem cells, pluripotent stem cells, and multipotent stem cells. In addition, based on biological origin, stem cells can be classified into embryonic stem cells and adult stem cells. Embryonic stem cells are derived from preimplantation embryos, developing fetal reproductive organs, and the like, whereas adult stem cells are derived from an individual organ, e.g., bone marrow, brain, liver, pancreas, or the like, of adults.

Totipotent stem cells are cells that have the ability to differentiate into any cell in an organism. Totipotency is maintained until the 8-cell stage after fertilization, occurring when a sperm fuses with an egg. Totipotent stem cells can be isolated and then transplanted into the uterus to develop into an intact individual.

Pluripotent stem cells are cells that have the capacity to differentiate into any types of cells and tissues which constitute ectoderm, mesoderm, or endoderm. Stem cells originate from an inner cell mass, located on the inside of a blastocyst that forms 4 to 5 days after fertilization, and the cells are called embryonic stem cells, which have the capacity to differentiate into multiple types of tissue cells, but have no capacity to develop into a new individual.

Multipotent stem cells are stem cells that have the capacity to differentiate only into specialized cell types in a specific tissue and organ where the stem cells are found.

Stem cells play a part in functions that maintain adult tissue homeostasis and induce regeneration in the event of tissue damage, as well as growth and development of respective tissue or organs during fetal, neonatal and adult periods. Particularly, adult stem cells are the general term for such tissue-specific multipotent cells. Adult stem cells are derived from existing cells, which are isolated from various organs of the human body and then developed into stem cells. Interestingly, although adult stem cells have general features to differentiate into specialized cell types of the tissue, recent studies have received attention by showing that adult stem cells can differentiate into various tissue cells, such as liver cells.

Preclinical and clinical research to apply stem cells to various diseases, such as cerebral infarctions, traumatic brain injuries, and musculoskeletal diseases, is underway. However, technologies pertaining to stem cell therapies currently have limitations, only focusing on isolating and culturing/proliferating stem cells, and injection thereof.

In addition, recent clinical research results showed that such stem cell therapies do not exhibit distinct effects yet.

There are several problems in applying therapeutic methods using stem cells to clinical trials. For example, tumor masses may be formed after engraftment of stem cells to organs, and cerebral infarction may occur due to an artery occlusion likely induced by the large size of stem cell itself. The stem cells easily move into the brain when the brain-blood vessel barrier is open as in an acute stage.

Perinatal brain injury (PBI) in preterm birth is associated with substantial injury and dysmaturation of white and gray matter, and can lead to severe neurodevelopmental deficits. Here, mesenchymal stromal cells (MSC) have been suggested to have neuroprotective effects in perinatal brain injury, in part through the release of extracellular vesicles like exosomes.

An exosome is a small vesicle with a membrane structure secreted from a variety of cell types. Exosomes have a diameter of about 30-100 nm. Exosomes derived from specific intracellular parts called multivesicular bodies (MVBs) and released and secreted to the outside of cells, instead of being directly detached and released from plasma membranes, by means of an electron microscope. That is, when fusion between multivesicular bodies and plasma membranes occurs, vesicles are released to the outside of cells. These vesicles are called exosomes. It is known that a variety of immunocytes including B lymphocytes, T lymphocytes, dendritic cells, platelets, macrophages, etc., tumor cells, and stem cells, as well as red blood cells, produce and secret exosomes during their lifespan. In particular, it is known that, since stem cell-derived exosomes contain nuclear components as well as receptors and proteins, the exosomes play roles in intercellular communication.

MSC exosomes applied prior ischemia very significantly prevent perinatal brain injury. MSC exosomes represent a promising strategy to prevent preterm PBI in human newborns. Their capacity to prevent gray and white matter alterations and especially their ability to improve long-term neurodevelopmental outcome as well as their feasibility to be administered in a minimally invasive yet effective, intranasal manner renders MSC exosomes in principle a solution for the problem. MSC-EVs exert immunomodulatory activities in many disease models including ischemic stroke, apparently in a comparable manner than MSCs. However, transferring this solution into the clinic and applying this manner on a wider scale is challenging for several reasons.

First of all, and primarily MSCs are functionally heterogenous. That means that not all MSCs are therapeutically active. It has been shown that the efficacy of MSCs for steroid-refractory acute GVHD associates with MSC donor age and a defined molecular profile. A significant survival benefit for patients treated with MSC derived from young (<10 years of age) compared to older (>10 years) MSC donors has observed.

MSCs are regularly polyclonal and undergo clonal selection procedures during their expansion. However, only a therapeutically active MSC can give rise to therapeutically active exosomes. Whether or not MSC's or exosomes derived therefrom are therapeutically active might depend on and require,
donor selection based on mechanistically defined potency assays,
analysis of early versus late passage,
analysis of MSC functionality,
analysis of senescent cell content and
analysis of pre-existing and acquired alloimmunization to MSC products and impact on transfusion biology.

Cryopreservation effects on viability and functionality may further define MSCs. Regarding the application of exosome it has been found that independent MSC-EV preparations differ in their potency. Even though the EVs from so called clonally expanded immortalized MSCs (ciMSCs) provide therapeutic activities, MSC source dependent differences remain.

It would therefore be of great importance to be able to supply a non-heterogenous and functionally active fraction of exosomes over and over again.

Such a fraction should ideally be producible in a GMP conform manner, it should be standardized, reproducible and of defined quality.

Any immune reaction by the recipient should be avoided.

### SUMMARY OF THE INVENTION

The present inventors have solved the problems mentioned above by providing the present invention which relates to a method of producing a clonal mesenchymal stem cell line capable of producing exosomes (MSC-derived extracellular vesicles) comprising the steps of, providing human induced pluripotent stem cells (hiPSCs), generating therefrom an immortalized clonal cell line of mesenchymal stem cells (IMSC), optionally characterizing the potential of the IMSCs to produce exosomes.

The invention also relates to a stem cell line produced according to the invention.

The invention relates to the cell line according to the invention, for use as a medicament.

The invention relates to the cell line according to the invention for use in treating a disease that is amenable to treatment with stem cell therapy.

The invention also relates to the use of a stem cell line according to the invention for producing EVs.

The present invention also relates to a method of producing a mesenchymal stem cell line capable of producing exosomes (MSC-derived extracellular vesicles) comprising the steps of, providing human induced pluripotent stem cells (hiPSCs), generating therefrom mesenchymal stem cells (MSCs), and optionally characterizing the potential of the MSCs to produce exosomes.

### Definitions

As used herein, the terms “MSC”, “MSCs”, “mesenchymal stromal cells”, “mesenchymal stem cells”, “hiPSC-derived MSCs” can interchangeably be used and refer to, if not otherwise stated or clear from the context, cells derived from human induced pluripotent stem cells (hiPSCs), wherein the cells possess characteristics or properties associated with mesenchymal stromal/stem cells. They can also be referred to “MSC-like cells” herein. Such characteristics comprise, but are not limited to, the expression (e.g. CD44, CD73, CD90, CD105) or absence (e.g. CD45, CD31, CD34, CD14, CD11b, CD79a or CD19, TRA 1-60, HLA-DR) of certain surface marker proteins, as described in more detail in another section of this patent/patent application. Furthermore, the cells are able to adhere to Laminin-coated and/or plastic plates, flasks or similar products used in cell culture. The cells also exhibit a “fibroblastoid” morphology, which refers to a fibroblast-like morphology such as, but not limited to, a spindle-shaped morphology.

The term “immortalized” refers to a process of genetically manipulating a cell or cells in order to avoid senescence and to enable proliferation for a limitless, or at least a prolonged period within in vitro cell culture. Examples of genes used for the genetic manipulation comprise hTERT, SV40T or p19^{ARF}.

As used herein, the term “coated” refers to cell culture plastic material, such as, but not limited to, plates or flasks, to which a cell culture matrix comprising peptides, e.g. recombinant Laminin-511 E8 protein fragments, was added in order to cover the plastic with these peptides. This supports adherence of cells. Other coating material known in the art, which is suitable to enable proliferation of the cells according to the invention, are also encompassed within the present invention.

If not stated otherwise, the term “MSC medium” or “MSCG medium” refers to the medium in which MSCs are cultured starting from e.g. day 6 after induction of hiPSC differentiation. Every medium, which is suitable for cultivation of hiPSC-derived MSCs, shall be understood to be encompassed within the present invention. However, serum-free medium is preferred.

“Xeno-free” as used herein means a formulation (e.g. cell culture medium) comprising human-derived components, but not components from other animals/species.

“Serum-free” as used herein means a formulation (e.g. cell culture medium) without serum. Instead, a well defined set of components is used. This enables standard and reproducible conditions and minimizes batch-to-batch variances.

“Conditioned medium” as used herein refers to the medium in which MSCs were grown and into which MSCs released EVs. However, it does not necessarily need to be MSC medium, it also encompasses other media (for example medium, which was optimized to yield high EV numbers).

“Clonal” as used herein means that a cell was derived from a pool of cells. If this single cell is expanded, a clonally expanded cell line is generated, which consists of cells that are all progenitors from one single cell.

“Passaging” relates to subculturing of cells and refers to a procedure which enables further propagation of the cells. Usually, this is done by reducing the number of cells per plate. Each time this procedure is performed, the passage number of the cells increases by one. Generation of a new cell line by e.g. genetic manipulation may lead to a restart of passage number counting from passage 0 (P0) or passage 1 (P1) again.

A “marker” as used herein relates to a molecule within or on a cell or EV, which can be detected and which reveals certain characteristics about the cell or EV. For example, the origin or the present metabolic or differentiation status or the type or content of cells or EVs can be determined using markers.

MSC-specific markers are markers, which are considered to define MSCs.

Non-MSC markers are markers, which are not considered to define MSCs.

Herein, the term “potency assay” is used synonymously with “functional assay”. It relates to an assay that tests for EV immunomodulatory activity or potential.

Herein, the term “immunomodulatory activity” is used synonymously with “immunomodulatory potential”. It relates to the potential of cells or EVs to modulate immune cells. For example, the activity and/or number of immune cells, such as, but not limited to, T cells, can be modulated.

“CD[No.]⁺” or “CD[No.]+” herein refers to cells or EVs expressing the respective protein or mRNA molecule encoding the respective protein, whereas “CD[No.]⁻” or “CD[No.]−” herein refers to cells or EVs not expressing the respective protein or mRNA molecule encoding the respective protein.

The addition of Penicillin-Streptomycin (Pen-Strep; PS) is considered optional in all media, even if not explicitly mentioned.

Herein, the terms “EV” and “exosome” are used synonymously.

### DETAILED DESCRIPTION OF THE INVENTION

The present invention relates to a method of producing a clonal mesenchymal stem cell line capable of producing exosomes (MSC-derived extracellular vesicles) comprising the steps of,

providing human induced pluripotent stem cells (hiPSCs),

generating therefrom an immortalized clonal cell line of mesenchymal stem cells (IMSC), optionally characterizing the potential of the IMSCs to produce exosomes.

The present invention also relates to a method of producing a mesenchymal stem cell line capable of producing exosomes (MSC-derived extracellular vesicles) comprising the steps of, providing human induced pluripotent stem cells (hiPSCs), generating therefrom mesenchymal stem cells (MSCs), and optionally characterizing the potential of the MSCs to produce exosomes. All methods described herein pertaining to immortalized and/or clonal MSCs can also be performed with non-immortalized and non-clonal MSCs, at least because the MSCs as produced by the methods of the present invention proliferate well for more than 70 passages (see FIG. 4), which is enough to perform the methods of the invention such as, but not limited to, EV preparations. Therefore, the invention also pertains to the respective methods when non-immortalized and non-clonal MSCs are used.

Mesenchymal stem cells have been used to treat various diseases. The therapeutic agent carried by these MSCs are the exosomes. Particular diseases are more suited than others for being targeted by such treatment. Such diseases are preferably “disease amenable to treatment with stem cell therapy”.

Bailo et al., 2004 could show that MSC-like cells derived from perinatal tissue show in vitro immunomodulatory potential. These cells could moreover be xenografted without rejection reactions (Bailo et al., 2004). Perinatal tissue, such as umbilical cord, are broadly available. It can be used to isolate cells, which often reveal high immunomodulatory properties exerted by the exosomes/EVs they produce.

Extracellular vesicles (EVs) are lipid bilayer-delimited particles that are naturally released from almost all types of cell and, unlike a cell, cannot replicate.

Herein, EVs and exosomes are used synonymously.

EVs range in diameter from near the size of the smallest physically possible unilamellar liposome (around 20-30 nanometers) to as large as 10 microns or more, although the vast majority of EVs are smaller than 200 nm. EVs according to size and synthesis route divided to Exosomes, microvesicles and apoptotic bodies. They carry a cargo of proteins, nucleic acids, such as miRNAs, mRNAs, small RNAs, large RNAs or DNA, lipids, metabolites, and even organelles from the parent cell. Most cells that have been studied to date are thought to release EVs, including some archaeal, bacterial, fungal, and plant cells that are surrounded by cell walls. A wide variety of EV subtypes have been proposed, defined variously by size, biogenesis pathway, cargo, cellular source, and function, leading to a historically heterogenous nomenclature including terms like exosomes and ectosomes.

Numerous functions of EVs have been established or postulated. The first evidence for the existence of EVs was enabled by the ultracentrifuge, the electron microscope, and functional studies of coagulation in the mid-20th century. A sharp increase in interest in EVs occurred in the first decade of the 21st century following the discovery that EVs could transfer nucleic acids such as RNA from cell to cell. Associated with EVs from certain cells or tissues, nucleic acids could be easily amplified as markers of disease and also potentially traced back to a cell of origin, such as a tumor cell. The discovery also implied that EVs could be used for therapeutic purposes, such as delivering nucleic acids or other cargo to diseased tissue. This growing interest was paralleled by formation of companies and funding programs focused on development of EVs as biomarkers or therapies of disease, the founding of an International Society for Extracellular Vesicles (ISEV), and establishment of a scientific journal devoted to the field, the Journal of Extracellular Vesicles.

Exosome biogenesis begins with pinching off of endosomal invaginations into the multivesicular body (MVB), forming intraluminal vesicles (ILVs). If the MVB fuses with the plasma membrane, the ILVs are released as “exosomes.” The first publication to use the term “exosome” for EVs presented it as a synonym for “micro-vesicle.” The term has also been used for EVs within specific size ranges, EVs separated using specific methods, or even all EVs.

“Disease amenable to treatment with stem cell therapy” as referred to herein means any procedures, conditions, disorders, ailments and/or illnesses which can be treated by the administration of stem cells and consequently herein by administration of the exosome they carry.

Such diseases include but are not limited to bone marrow, skin, heart, and corneal transplantation, graft versus host disease, hepatic and renal failure, lung injury, bronchopulmonary dysplasia, rheumatoid arthritis, treatment of autoimmune diseases such as Crohn's disease, ulcerative colitis, multiple sclerosis, lupus and diabetes; prevention of allograft rejection, neurological disorders and cardiovascular medicine; as well as Acute lymphoblastic leukemia (ALL), Acute myeloid leukemia (AML), Burkitt's lymphoma, Chronic myeloid leukemia (CML), Juvenile myelomonocytic leukemia (JMML), Non-Hodgkin's lymphoma Hodgkin's lymphoma, Lymphomatoid granulomatosis, Myelodysplastic syndrome (MDS), Chronic myelomonocytic leukemia (CMML), Bone Marrow Failure Syndromes, Amegakaryocytic thrombocytopenia, Autoimmune neutropenia (severe), Congenital dyserythropoietic anemia, Cyclic neutropenia, Diamond-Blackfan anemia, Evan's syndrome, Fanconi anemia, Glanzmann's disease, Juvenile dermatomyositis, Kostmann's syndrome, Red cell aplasia, Schwachman syndrome, Severe aplastic anemia, Congenital sideroblastic anemia, Thrombocytopenia with absent radius (TAR syndrome), Dyskeratosis congenital, Blood Disorders, Sickle-cell anemia (hemoglobin SS), HbSC disease, Sickle βo Thalassemia, α-thalassemia major (hydrops fetalis), β-thalassemia major (Cooley's anemia), β-thalassemia intermedia, E-βo thalassemia, E-β+ thalassemia, Metabolic Disorders, Adrenoleukodystrophy Gaucher's disease (infantile), Metachromatic leukodystrophy, Krabbe disease (globoid cell leukodystrophy), Gunther disease, Hermansky-Pudlak syndrome, Hurler syndrome, Hurler-Scheie syndrome, Hunter syndrome, Sanfilippo syndrome, Maroteaux-Lamy syndrome, Mucolipidosis Type II, III, Alpha mannosidosis, Niemann Pick Syndrome, type A and B, Sandhoff Syndrome, Acute liver failure, Tay-Sachs Disease, Batten disease (inherited neuronal ceroid lipofuscinosis), Lesch-Nyhan disease, Immunodeficiencies, Ataxia telangiectasia, Chronic granulomatous disease, DiGeorge syndrome, IKK gamma deficiency, Immune dysregulation polyendocrineopathy, X-linked Mucolipidosis, Type II, Myelokathexis X-linked immunodeficiency, Severe combined immunodeficiency, Adenosine deaminase deficiency, Wiskott-Aldrich syndrome, X-linked agammaglobulinemia, X-linked lymphoproliferative disease, Omenn's syndrome, Reticular dysplasia, Thymic dysplasia, Leukocyte adhesion deficiency, Other Osteopetrosis, Langerhans cell histiocytosis, Hemophagocytic lymphohistiocytosis, Acute & Chronic Kidney Disease, Acute Kidney failure, Alzheimer's disease, Anti-Aging, Arthritis, Asthma, Cardiac Stem Cell Therapy, Cerebral Infarction (Stroke), Cerebral Palsy (Stroke), Chronic Obstructive Pulmonary Disease (COPD), Congestive Heart Failure, Diabetes Mellitus (Type I & II), Fibromyalgia, Immune Deficiencies, Ischemic Heart Disease, Lupus, Multiple Sclerosis, Myocardial/Cardiac Infarction, Heart failure, Osteoarthritis, Osteoporosis, Parkinson's Disease, Peripheral Arterial Disease, Rheumatoid Arthritis, Stem Cell Therapy in Plastic Surgery, Traumatic Brain Injury, Neurological Diseases, pulmonary hypertension, pulmonary arterial hypertension, various forms of brain damage and spinal cord injury in newborns, children, and adults such as, but not limited to, white matter brain damage, periventricular leukomalacia, intracranial hemorrhage, cerebral hemorrhage, arterial stroke, cerebral venous thrombosis, traumatic brain injury (TBI), brain concussion, spinal cord injury (SCI), neonatal encephalopathy, cerebral palsy, infectious brain damage, encephalitis, meningitis, amyotrophic lateral sclerosis (ALS), Alzheimer dementia, Parkinson, and Multiple sclerosis.

EVs can also improve brain plasticity synaptogenesis and connectome formation (Jensen, 2019, Jensen A. Pediatric Stroke and Cell-Based Treatment-Pivotal Role of Brain Plasticity. J Stem Cell Res Transplant. 2019; 6(1): 1029.), thus improving motor-and cognitive function and behavior. EVs can be administered intravenously, intra-arterially, intraperitoneally, intranasally, intrathecally, in joints (intra-arthricularly), endoscopically or directly into the organs, such as, but not limited to, the eye.

Prior to characterizing the potential of the IMSCs exosomes may be isolated from these cells. This can be done by applying a standardized PEG precipitation protocol involving a final ultracentrifugation step (Borger et al., 2020; Kordelas et al., 2014; Ludwig et al., 2018), EVs were either prepared from supernatants harvested from cultures of different donor derived MSCs or of independent propagations of the same MSC batches.

Polyethylene glycol/polymer-based EV enrichment Precipitation by “salting out” with polyethylene glycol (PEG) or other polymers is an effective way to reduce volume and thus enrich EVs in a reproducible and scalable manner. While ultracentrifugation can be performed for only up to approximately 500 mL culture medium per run (depending on rotor and buckets), the lower-speed centrifugation required to pellet PEG precipitates can be done for up to several litres per run. Each run is also shorter for PEG precipitates. Because of abundant co-precipitates, however, PEG-precipitated EVs should not be considered pure preparations. Also, removal of PEG and other contaminants by wash steps and UC repelleting may be necessary for some applications. Nevertheless, PEG-precipitated MSC-sEVs (sEVs are small extracellular vesicles of about 50-200 nM in diameter) have already been used in a clinical investigation, and MSC-sEVs concentrated by PEG exerted the same effects in an ischaemic stroke model as corresponding cells. Thus, the procedure does not appear to interfere with MSC-sEV activity, and co-isolated materials do not appear to negatively affect sEV function.

Size-based fractionation methods such as size exclusion chromatography (SEC) and tangential flow filtration (TFF) have gained increasing recognition and adoption as GMP-compatible and highly scalable technologies by researchers. These methods are faster and easier to implement than legacy methods, while at the same time producing EVs of comparable or superior purity and/or functional activity. It is one of the preferred methods herein. Conditioned media with high protein content (e.g. serum-or hPL-supplemented media) may clog pores, especially in the case of fibrin formation from concentrated, unprocessed hPL. In this case, clotting can be induced in advance and clotted components removed; however, pre-processing serum or hPL may also change MSC-supportive properties. Apart from these considerations, these methods are considered scalable and time efficient.

In a preferred embodiment the method of the invention relates to a method of producing an immortal clonal mesenchymal stem cell line capable of producing exosomes (MSC-derived extracellular vesicles) comprising the steps of, i) providing human induced pluripotent stem cells (hiPSCs), ii) generating therefrom an immortalized clonal cell line of mesenchymal stem cells (IMSC), iii) characterizing the potential of the IMSCs to produce exosomes, wherein the exosomes display a surface marker selected from CD63 and CD9 and/or, wherein the immortalized clonal cell line of mesenchymal stem cells (IMSC) are characterized by their capability to form fat or bone cells.

Not all the immortal clonal mesenchymal stem cell lines will be capable of producing exosomes of the desired quality. Hence, the invention relates not just to a method of making such immortal clonal mesenchymal stem cell lines capable of producing exosomes but preferably in selecting those cell lines which display EVs of a desired quality. The surface markers CD63 and CD9 are one way of identifying these, their capability to form fat or bone cells is another way.

Preferably, the EVs have a density as determined by density gradient centrifugation of 1.13 g per mL to 1.19 g per mL.

Preferably, the hiPSCs have a homozygous leukocyte antigen (HLA) haplotype (HLA-homo HP).

Preferably, the hiPSCs are generated by means of one or more Yamanaka factors such as but not limited to Myc, Oct3, Oct4, Sox2 and Klf4 and stem from preferably umbilical cord blood tissue.

Cells of lesser potency can be, but are not limited to adult stem cells, tissue specific progenitor cells, primary or secondary cells. An adult stem cell is an undifferentiated cell found throughout the body after embryonic development. Adult stem cells multiply by cell division to replenish dying cells and regenerate damaged tissue.

Adult stem cells have the ability to divide and create another cell like itself and also divide and create a cell more differentiated than itself. Even though adult stem cells are associated with the expression of pluripotency markers such as Rex 1, Nanog, Oct4 or Sox2, they do not have the ability of pluripotent stem cells to differentiate into the cell types of all three germ layers. Adult stem cells have a limited potency to self-renew and generate progeny of distinct cell types.

Without limitation, an adult stem cell can be a hematopoietic stem cell, a cord blood stem cell, a mesenchymal stem cell, an epithelial stem cell, a skin stem cell or a neural stem cell. A tissue specific progenitor refers to a cell devoid of self-renewal potential that is committed to differentiate into a specific organ or tissue.

A primary cell includes any cell of an adult or fetal organism apart from egg cells, sperm cells and stem cells. Examples of useful primary cells include, but are not limited to, skin cells, bone cells, blood cells, cells of internal organs and cells of connective tissue.

A secondary cell is derived from a primary cell and has been immortalized for long-lived in vitro cell culture.

A “cord blood stem cell” refers to an adult stem cell that resides in cord blood and is characterized by a lesser potency to self-renew and differentiate than a pluripotent stem cell.

The term “transfection” or “transfecting” is defined as a process of introducing nucleic acid molecules to a cell by non-viral or viral-based methods. Non-viral methods of transfection include any appropriate transfection method that does not use viral DNA or viral particles as a delivery system to introduce the nucleic acid molecule into the cell.

Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral based methods of transfection any useful viral vector may be used in the methods described herein.

Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell.

Such a selection advantage may be a resistance towards a certain toxin that is presented to the cell. Expression of a transfected gene can further be accomplished by transposon-mediated insertion into to the host genome. During transposon-mediated insertion the gene is positioned between two transposon linker sequences that allow insertion into the host genome as well as subsequent excision.

An “OCT4 protein” as referred to herein includes any of the naturally-occurring forms of the Octomer 4 transcription factor, or variants thereof that maintain Oct4 transcription factor activity (e.g. within at least 50%, 80%, 90%, 95%, 96%, 97%, 98%, 99% or 100% activity compared to Oct4). In some embodiments, variants have at least 90%, 95%, 25 96%, 97%, 98%, 99% or 100% amino acid sequence identity across the whole sequence or a portion of the sequence (e.g. a 50, 100, 150 or 200 continuous amino acid portion) compared to a naturally occurring Oct4 polypeptide. In other embodiments, the Oct4 protein is the protein as identified by the NCBI reference gi:42560248 corresponding to isoform 1, gi:116235491 and gi:291167755 corresponding to isoform 2.

A “Sox2 protein” as referred to herein includes any of the naturally-occurring forms of the Sox2 transcription factor, or variants thereof that maintain Sox2 transcription factor activity (e.g. within at least 50%, 80%, 90%, 95%, 96%, 97%, 98%, 99% or 100% activity compared to Sox2). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Sox2 polypeptide. In other embodiments, the Sox2 protein is the protein as identified by the NCBI reference gi:28195386.

A “KLF4 protein” as referred to herein includes any of the naturally-occurring forms of the KLF4 transcription factor, or variants thereof that maintain KLF4 transcription factor activity (e.g. within at least 50%, 80%, 90%, 95%, 96%, 97%, 98%, 99% or 100% activity compared to KLF4). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KLF4 polypeptide.

In other embodiments, the KLF4 protein is the protein as identified by the NCBI reference gi:194248077.

A “cMYC protein” as referred to herein includes any of the naturally-occurring forms of the cMyc transcription factor, or variants thereof that maintain cMyc transcription factor activity (e.g. within at least 50%, 80%, 90%, 95%, 96%, 97%, 98%, 99% or 100% activity compared to cMyc). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cMyc polypeptide (e.g. SEQ ID NO:6). In other embodiments, the cMyc protein is the protein as identified by the NCBI reference gi:71774083.

A “NANOG protein” as referred to herein includes any of the naturally-occurring forms of the Nanog transcription factor, or variants thereof that maintain Nanog transcription factor activity (e.g. within at least 50%, 80%, 90%, 95%, 96%, 97%, 98%, 99% or 100% 25 activity compared to Nanog). In some embodiments, variants have at least 90%, 95%, 96%, 97%, 98%, 99% or 100% amino acid sequence identity across their whole sequence or a portion of the sequence (e.g. a 50, 100, 150 or 200 continuous amino acid portion) compared to the naturally occurring Nanog polypeptide. In other embodiments, the Nanog protein is the protein as identified by the NCBI reference gi: 153945816.

A “LIN28 protein” as referred to herein includes any of the naturally-occurring forms of the Lin28 transcription factor, or variants thereof that maintain Lin28 transcription factor activity (e.g. within at least 50%, 80%, 90%, 95%, 96%, 97%, 98%, 99% or 100% activity compared to Lin28). In some embodiments, variants have at least 90%, 95%, 96%, 97%, 98%, 99% or 100% amino acid sequence identity across their whole sequence or a portion of the sequence (e.g. a 50, 100, 150 or 200 continuous amino acid portion) compared to the naturally occurring Lin28 polypeptide.

In other embodiments, the Lin28 protein is the protein as identified by the NCBI reference gi: 13375938.

Allowing the transfected cord blood stem cell to divide and thereby forming the induced pluripotent stem cell may include expansion of the cord blood stem cell after transfection, optional selection for transfected cells and identification of pluripotent stem cells. Expansion as used herein includes the production of progeny cells by a transfected cord blood stem cell in containers and under conditions well known in the art. Expansion may occur in the presence of suitable media and cellular growth factors. Cellular growth factors are agents which cause cells to migrate, differentiate, transform or mature and divide. They are polypeptides which can usually be isolated from various normal and malignant mammalian cell types. Some growth factors can also be produced by genetically engineered microorganisms, such as bacteria (E.coli) and yeasts. Cellular growth factors may be supplemented to the media and/or may be provided through co-culture with irradiated embryonic fibroblasts that secrete such cellular growth factors. Examples of cellular growth factors include, but are not limited to FGF, bFGF2, and EGF.

Where appropriate the expanding transfected cord blood stem cell may be subjected to a process of selection. A process of selection may include a selection marker introduced into a cord blood stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected cord blood stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin, which no longer inhibits expansion and causes cell death of a transfected cord blood stem cell. Upon exposure to a toxin a cell lacking a selection marker may be eliminated and thereby precluded from expansion.

Identification of the induced pluripotent stem cell may include but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

As afore mentioned, the cord blood stem cell provided in the methods herein may be transfected with a nucleic acid encoding a OCT4 protein and a nucleic acid encoding a SOX2 protein. In some embodiments, the cord blood stem cell is not transfected with an additional nucleic acid encoding a cMYC protein, a LIN28 protein, a NANOG protein or a KLF4 protein. In some embodiments, the nucleic acid encoding an OCT4 protein forms part of a plasmid and the nucleic acid encoding a SOX2 protein forms part of a plasmid. In another embodiment, the nucleic acid encoding an OCT4 protein and the nucleic acid encoding a SOX2 protein form part of the same plasmid. In one embodiment, the nucleic acid encoding an OCT4 protein forms part of a first plasmid and the nucleic acid encoding a SOX2 protein forms part of a second plasmid. In another aspect, a method for preparing an induced pluripotent stem cell is provided. The method includes transfecting a cord blood stem cell with a nucleic acid encoding an OCT4 protein to form a transfected cord blood stem cell. The transfected cord blood stem cell is allowed to divide thereby forming the induced pluripotent stem cell.

In one embodiment, the cord blood stem cell is not transfected with an additional nucleic acid encoding a cMYC protein, a LIN28 protein, a NANOG protein or a KLF4 protein.

In some embodiments, the cord blood stem cell used in the methods provided herein expresses a CD133 antigen.

A “CD133 antigen” refers to a five transmembrane domain glycoprotein, which is 120 kilo Dalton in size. A CD133 antigen may be expressed by adult stem cells and progenitor cells. The CD133 antigen is also known was PROML1, AC133, hematopoietic stem cell antigen, hProminin, prominin-like 1, prominin, RP41, MCDR2, STGD4, CORD 12 or MSTP061. In some embodiments, the CD 133 antigen is the protein encoded by the gene identified by the NCBI reference gi:225690512.

In some embodiments, the cord blood stem cell used in the methods provided herein is derived from fresh cord blood.“

Fresh cord blood” is blood derived from the umbilical cord of a neonate, which is returned to the neonatal circulation if the umbilical cord is not prematurely clamped. Fresh cord blood as referred to herein is not cryopreserved after isolation from the umbilical cord.

The term “cryoconservation” refers to the process of freezing biological material such as cord blood using liquid nitrogen thereby conserving the biological material for long time periods. In other embodiments, the cord blood stem cell used in the methods provided herein is derived from frozen cord blood. Frozen cord blood is blood derived from the umbilical cord of a neonate that has been cryo-conserved prior to being processed according to the methods provided herein.

### An Induced Pluripotent Stem Cell

In another aspect, an induced pluripotent stem cell prepared in accordance with the methods herein is provided. The methods described above in the section entitled “Methods of Preparing Induced Pluripotent Stem Cells from Cord Blood” are equally applicable to an induced pluripotent stem cell as provided herein.

### Cord Blood Stem Cells

In one aspect, a cord blood stem cell including a nucleic acid encoding an OCT4 protein (e.g. an exogenous nucleic acid encoding an OCT4 protein or a recombinant nucleic acid encoding an OCT4 protein) and a nucleic acid encoding a SOX2 protein (e.g. an exogenous nucleic acid encoding an SOX2 protein or a recombinant nucleic acid encoding an SOX2 protein) is provided. The term “exogenous” in reference to a nucleic acid encoding a protein as used herein means not naturally occurring in the cell in which it is found (e.g. a cord blood cell). In some embodiments, the nucleic acid encoding an OCT4 protein forms part of a plasmid and the nucleic acid encoding a SOX2 protein forms part of a plasmid. In another embodiment, the nucleic acid encoding an OCT4 protein and the nucleic acid encoding a SOX2 protein form part of the same plasmid. In one embodiment, the nucleic acid encoding an OCT4 protein forms part of a first plasmid and the nucleic acid encoding a SOX2 protein forms part of a second plasmid. In some embodiments, the cord blood stem cell does not include nucleic acids encoding other transcription factors known to be useful in iPS cell formation, such as a nucleic acid encoding a cMYC protein (e.g. an exogenous nucleic acid encoding a cMYC protein or a recombinant nucleic acid encoding a cMYC protein), a nucleic acid encoding a LIN28 protein (e.g. an exogenous nucleic acid encoding a LIN28 protein or a recombinant nucleic acid encoding a LIN28 protein), a nucleic acid encoding a NANOG protein (e.g. an exogenous nucleic acid encoding a NANOG protein or a recombinant nucleic acid encoding a NANOG protein) and/or a nucleic acid encoding a KLF4 protein (e.g. an exogenous nucleic acid encoding a KLF4 protein or a recombinant nucleic acid encoding a KLF4 protein).

In other embodiments, the cord blood stem cell consists essentially of a nucleic acid encoding an OCT4 protein (e.g. an exogenous nucleic acid encoding an OCT4 protein or a recombinant nucleic acid encoding an OCT4 protein) and a nucleic acid encoding a SOX2 protein (e.g. an exogenous nucleic acid encoding an SOX2 protein or a recombinant nucleic acid encoding an SOX2 protein). Where a cord blood stem cell “consists essentially of” a nucleic acid encoding an OCT4 protein and a nucleic acid encoding a SOX2 protein, the cord blood stem cell does not include nucleic acids encoding other transcription factors known to be useful in iPS cell formation, such as a nucleic acid encoding a cMYC protein (e.g. an exogenous nucleic acid encoding a cMYC protein or a recombinant nucleic acid encoding a cMYC protein), a nucleic acid encoding a LIN28 protein (e.g. an exogenous nucleic acid encoding a LIN28 protein or a recombinant nucleic acid encoding a LIN28 protein), a nucleic acid encoding a NANOG protein (e.g. an exogenous nucleic acid encoding a NANOG protein or a recombinant nucleic acid encoding a NANOG protein) and/or a nucleic acid encoding a KLF4 protein (e.g. an exogenous nucleic acid encoding a KLF4 protein or a recombinant nucleic acid encoding a KLF4 protein). In some embodiments, the cord blood stem cell does not include nucleic acids encoding other transcription factors (e.g. other exogenous nucleic acids encoding a transcription factor or other recombinant nucleic acids encoding a transcription factor). In other embodiments, the cord blood stem cell does not include nucleic acids encoding other protein expressing genes (e.g. other exogenous nucleic acids encoding a protein or other recombinant nucleic acids encoding a protein).

In another aspect, a cord blood stem cell including a nucleic acid encoding an OCT4 protein (e.g. an exogenous nucleic acid encoding an OCT4 protein or a recombinant nucleic acid encoding an OCT4 protein) is provided. In other embodiments, the cord blood stem cell consists essentially of a nucleic acid encoding an OCT4 protein (e.g. an exogenous nucleic acid encoding an OCT4 protein or a recombinant nucleic acid encoding an OCT4 protein).

Where a cord blood stem cell “consists essentially of” a nucleic acid encoding an OCT4 protein, the cord blood stem cell does not include nucleic acids encoding other transcription factors known to be useful in iPS cell formation, such as a nucleic acid encoding a cMYC protein (e.g. an exogenous nucleic acid encoding a cMYC protein or a recombinant nucleic acid encoding a cMYC protein), a nucleic acid encoding a LIN28 protein (e.g. an exogenous nucleic acid encoding a LIN28 protein or a recombinant nucleic acid encoding a LIN28 protein), a nucleic acid encoding a NANOG protein (e.g. an exogenous nucleic acid encoding a NANOG protein or a recombinant nucleic acid encoding a NANOG protein) and/or a nucleic acid encoding a KLF4 protein (e.g. an exogenous nucleic acid encoding a KLF4 17 protein or a recombinant nucleic acid encoding a KLF4 protein). In some embodiments, the cord blood stem cell does not include nucleic acids encoding other transcription factors (e.g. other exogenous nucleic acids encoding a transcription factor or other recombinant nucleic acids encoding a transcription factor). In other embodiments, the cord blood stem cell does not include nucleic acids encoding other protein expressing genes (e.g. other exogenous nucleic acids encoding a protein or other recombinant nucleic acids encoding a protein).

In some embodiments, the cord blood stem cell expresses a CD133 antigen. In other embodiments, the cord blood stem cell is derived from fresh cord blood. In some embodiments, the cord blood stem cell is derived from frozen cord blood.

### Induced Pluripotent Stem Cells

In one aspect, a method for producing a human somatic cell is provided. The method includes contacting an induced pluripotent stem cell with cellular growth factors. The induced pluripotent stem cell is allowed to divide, thereby forming the human somatic cell. The induced pluripotent stem cell is allowed to divide in the presence of appropriate media and cellular growth factors. Examples for cellular growth factors include, but are not limited to, SCF, GMCSF, FGF, TNF, IFN, EGF, IGF and members of the interleukin family. The induced pluripotent stem cell is prepared in accordance with the methods provided by the present invention. In some embodiments, the induced pluripotent stem cell is prepared by a process including the steps of transfecting a cord blood stem cell with a nucleic acid encoding an OCT4 protein and a nucleic acid encoding a SOX2 protein to form a transfected cord blood stem cell. The transfected cord blood stem cell is allowed to divide thereby forming the induced pluripotent stem cell. In another embodiment, the induced pluripotent stem cell is prepared by a process including the steps of transfecting a cord blood stem cell with a nucleic acid encoding an OCT4 protein to form a transfected cord blood stem cell. The transfected cord blood stem cell is allowed to divide thereby forming the induced pluripotent stem cell.

Preferably, the method of the inventions comprises transfecting a cord blood stem cell with a nucleic acid encoding an OCT4 protein and a nucleic acid encoding a SOX2 protein to form a transfected cord blood stem and allowing said transfected cord blood stem cell to divide thereby forming said induced pluripotent stem cell.

Preferably, the generation of immortalized clonal cell line of mesenchymal stem cells (IMSC) is done by means of a tissue specific media, such as a cardiomyogenic medium (CARM) and optionally a specific p38-MAPK inhibitor, wherein after generation the resulting IMSC expresses one or more of the following markers selected from the group of CD29, CD44, CD73, CD90, and CD105.

In a preferred embodiment, hiPSCs are differentiated to MSCs by firstly using a xeno-free medium and a WNT activator. From 6 days after induction of differentiation onwards, the cells are cultivated in defined, xeno-and serum-free medium. This minimizes heterogeneity that may occur due to different serum batches with unknown concentrations of the ingredients, such as, but not limited to, growth factors. The details of this preferred method of hiPSC-derived MSC generation are described in example 6.2.1.

Although not preferred, differentiation of hiPSCs to MSCs can also be performed by using knockout serum replacement medium.

From 6 days after induction of differentiation onwards, hiPSC-derived MSCs can be cultured in low glucose DMEM +10% fetal-calf serum (FCS) or 10% human platelet lysate (hPL)+optionally supplemented with 1% Penicillin/Streptomycin. However, as mentioned above, it is preferred that MSCs are cultured in defined, xeno-and serum-free medium such as, but not limited to, StemMacs MSC Expansion Media (Miltenyi; cat. no.: 130-104-182) or Sartorius.

In an alternative embodiment, hiPSC-derived MSCs can be cultured in low glucose DMEM+10% fetal-calf serum (FCS) or 10% human platelet lysate (hPL)+optionally supplemented with 1% Penicillin/Streptomycin from day 1 or from day 2 or from day 3 or from day 4 or from day 5 or from day 7 or from day 8 or from day 9 or from day 10 after induction of differentiation onwards.

Several WNT activators known in the art can be used, for example, lithium chloride (2 mM), SB-216763 (5.7 M), BIO (0.48 μM), or CHIR99021 (CHIR; STEMCELL Technologies). The concentration of CHIR99021 can be 3.5 μM or 3.8 μM or 4 μM or 4.3 μM or 4.5 μM or 4.8 μM or 4.9 μM or 5.3 μM or between 3.5 μM and 5.3 μM or between 3.8 μM and 4.9 μM or between 3.5 μM and 4.8 μM. Preferably, CHIR99021 is used as a WNT activator at a concentration of 4 μM.

hiPSCs can be seeded at different cell densities (2.500 or 5.000 or 10.000 or 20.000 or 40.000 or 100.000 or 200.000 cells/cm²) without noticeable difference in differentiation outcome. Preferably, 100.000 cells are seeded.

Preferably, the plates are pre-coated for at least 1 hour at 37° C. using 2 ml XF medium+PS+Y, supplemented with 3 μl iMatrix-511 prior to seeding of hiPSCs.

WNT activation can last for 4 days or 5 days or 6 days or 7 days. Preferably, WNT activation and thus induction of differentiation lasts for 6 days.

Starting from day 4 or day 5 or day 6 or day 7 onwards, hiPSC-derived MSCs are cultured in MSC medium (also referred to MSCG medium). MSC medium can also relate to low glucose DMEM+10% FCS+optionally Pen-Strep diluted 1:100. MSC medium can also relate to low glucose DMEM+10% hPL+optionally Pen-Strep diluted 1:100. MSC medium can also relate to high glucose DMEM+10% FCS+optionally Pen-Strep diluted 1:100. MSC medium can also relate to high glucose DMEM+10% hPL+optionally Pen-Strep diluted 1:100. MSC medium preferably relates to serum-free medium. One example for such a medium is StemMacs MSC Expansion Media (Miltenyi Biotec)+700 μl/50 ml medium StemMacs MSC Expansion Media Supplement (Miltenyi Biotec)+optionally Pen-Strep diluted 1:100.

Surprisingly, cells grew better on Laminin-coated plates than on plastic at passage 1. From passage 2 onwards, cells grew equally well on Laminin-coated, as well as on plastic plates.

hiPSC-derived MSCs are preferably passaged at a ratio of 1:2 for the first 3 passages. Alternatively, cells can be passaged at a ratio of 1:2 for the first 2 passages or at a ratio of 1:3 for the first 3 passages.

A WNT inhibitor, such as C59 can be used for one, two, three, four, five, six or more days following WNT activation. If used, a four day treatment is preferred. Transient usage of a WNT inhibitor can improve expression of MSC-specific markers, such as CD105 and CD73, in later passages (e.g. P18). Furthermore, transient usage of a WNT inhibitor can maintain lesser population doubling times in later passages.

The induced MSCs express one or more of the following MSC-specific markers selected from the group comprising CD29, CD44, CD73, CD90, and CD105. The MSCs do not or only weakly express any of the following non-MSC markers selected from the group comprising CD31, CD34, CD45, CD14, TRA 1-60 and HLA-DR. Technically, the presence of MSC-and non-MSC markers can be analysed by methods comprising, but not limited to, western blotting, enzyme-linked immunosorbent assays, or flow cytometry.

The induced MSCs exhibit a fibroblastoid morphology and adhere to plastic or Laminin-coated plates or flasks (FIGS. 1 and 2). While adherence to plastic appears to be more efficient for later passages (see for example P1 cells of FIG. 1 compared to older passages as seen in FIG. 2), both Laminin-and plastic adherence are characteristics of MSCs.

In a preferred embodiment, the induced MSCs are expandable over at least 5, or at least 10, or at least 15, or between 4 and 38, or between 7 and 45, or between 9 and 69 passages.

Cell density during initiation of differentiation (the first 6 days during which a WNT activator is used) does not influence hiPSC differentiation potential towards MSCs (FIG. 3).

hiPSC-derived MSCs show constantly high proliferation capacities even until late passages (>70 days after induction), independently of the MSC medium (low glucose DMEM, supplemented with FCS or hPL (FIG. 4A and 4C) or serum-free medium (FIG. 4B)) and the hiPSC cell line (R23, R25, R26-6) used (FIG. 4). All of these cells adhere to plastic or Lamnin-coated plates/flasks, express the MSC-specific markers and do not express the non-MSC markers. Instead of low-glucose medium, high-glucose medium can be used after WNT activation, but high-glucose medium is preferably used in combination with hPL, rather than FCS. MSC media can be changed during culture, e.g. MSCs derived and maintained in serum-free medium can be transferred to and grown in low-glucose DMEM+10% hPL whenever needed.

MSCs derived from different hiPSC lines (R23, R25 and R26-6) all express the key MSC marker proteins CD44, CD73 and CD90 and lowly express CD105 (FIG. 5), but do not express non-MSC-/other lineage marker proteins (FIG. 6), which comprise TRA 1-60, CD31, CD45, CD14, CD34, HLA-DR. Compared to other markers, the extent of CD105 expression and the number of cells expressing CD105 can be variable and may depend on different factors, such as, but not limited to, the passage number, culturing conditions, such as the presence or absence of serum, or the origin of the cells from which MSCs are derived. However, at least the expression of the other three MSC-specific markers was constantly high from low to high passage MSCs (FIG. 7). Furthermore, cultivation of MSCs under serum-free, defined media conditions express the key MSC marker proteins CD44, CD73, CD90 and CD105 (FIG. 8), but do not express non-MSC-/other lineage marker proteins (FIG. 9), which comprise TRA 1-60, CD31, CD45, CD14, CD34, HLA-DR.

mRNA expression analyses using quantitative real-time PCR (qRT-PCR) reveal, that hiPSC-derived MSCs do not express pluripotency markers or the epithelial marker CDH1, but do express the mesenchymal marker CDH2 (FIG. 10) and CD73, CD44 or CD105, whereas other mesodermal cell types derived from iPSCs (endothelial progenitor cells (iPSC-EPCs), hematopoietic stem cells (iPSC-HSCs) and cardiomyocytes (iPSC-CMs) only weakly express the MSC markers, if at all (FIG. 11). Other hiPSC-derived mesodermal cell types do, however, express their cell type specific markers, whereas hiPSC-MSCs do not (FIG. 12), demonstrating that the hiPSC-derived MSCs of the present invention are devoid of residual pluripotent stem cells, endothelial, hematopoietic or cardiac contaminants and instead express the MSC-specific markers.

The MSCs of the present invention can further be differentiated into osteoblasts, adipocytes and chondrocytes (FIG. 13), highlighting hat the defined serum-free media derived MSCs are bona fide MSCs.

Extracellular vesicles can be isolated from conditioned medium as described for instance in example 9.2. EVs collected from conditioned medium of different MSC passages express the EV surface markers CD9, CD63 and CD81 (FIG. 14). EVs, especially EVs of later passages, also exhibit immunomodulatory activities towards CD4⁺ and CD8⁺ T cells, as revealed by analysis of the T cell activation markers CD25 and CD54 by a mixed lymphocyte reaction (mdMLR) assay (FIG. 15).

To identify MSC-sEV-specific antigens, the presence of MSC surface antigens in published MSC-(s)EV proteome databases can be investigated. These revealed three MSC surface antigens from the ISCT minimal criteria CD73, CD90 and CD105.

These findings support using the MSC positive and negative surface antigens listed above to assess the identity and purity of the cellular source of an MSC-sEV preparation. Technically, the presence of MSC-and non-MSC-sEV markers can be analysed by methods including Western blotting, enzyme-linked immunosorbent assays, classical flow cytometry of bead-captured EVs, or advanced flow cytometry at the single EV level.

Preferably, the MSC cellular origin of an sEV preparation is identified by the presence of MSC markers, CD44, CD73, CD90 and CD105, and the absence of CD14, CD31, CD34, CD45, HLA-DR and CD11b. It is, however, recognized that it may not be practical for all MSC-sEV preparations to be devoid of non-MSC markers, especially if the MSCs are cultured in the presence of supplements such as hPL or serum. Preferably relative abundance of MSC versus non-MSC markers in an MSC-sEV population helps to assess the relative ratio of MSC-sEV to non-MSC-sEVs and is useful in calibrating comparison between different MSC-sEV preparations.

It is important to distinguish EV preparations from other particle preparations. To distinguish an sEV preparation from other biological nanoparticle preparations, the following quantifiable metrics can be used: ratio of specific membrane lipids to proteins and ratio of sphingomyelin to phosphatidylcholine.

In addition to the ratiometric approaches above, absolute quantitation of sEVs is preferred. Unfortunately, with the exception of volumetric cryoelectron microscopy (which also unambiguously reveals lipid membrane vesicles), identifying particles as EVs is difficult. Nevertheless, progress in single EV analysis by methods such as fluorescence-augmented NTA or nano-flow is promising to the extent that lipid and protein labelling can be included.

In a preferred embodiment after purification of EVs all proteins and RNA are anchored in or encapsulated within a lipid bilayer membrane. Such a result is preferred. The isolation will be deemed successful if that is achieved.

For sEVs with a size range of 50-200 nm and a specific membrane lipid to protein ratio, the number of sEVs is limited by the amount of membrane lipids or proteins. Hence, if the number of sEVs is known and measured as lipid membrane vesicles the number of sEVs per unit membrane lipids or proteins reflects the degree of purity. Furthermore, loosely associated factors that contribute to MSC-sEV biological activities are removed by stringent purification, resulting in a reduction or even loss of therapeutic activities in the disease models of interest. This is to be avoided.

sEVs integrity is preferred. EV integrity is synonymous with lipid membrane integrity. Electron microscopy provides irrefutable evidence for the presence of intact membrane vesicles. Membrane integrity may be assayed using proteins that are tethered to a membrane lipid, such as GM1 ganglioside. GM1 is preferred as a tool. GM1 gangliosides are highly enriched in MSC-sEVs and are bound with high affinity by cholera toxin B chain (CTB). CTB-binding sEVs are also enriched in CD81, and this association can be readily assayed by ELISA. Disruption of vesicles by homogenization disrupts the association between CD81 and CTB binding, paralleled by loss of function (e.g. cardioprotective activity) and reduced CD81 in CTB-bound sEVs. Thus, the level of CTB associated CD81 in a preparation provides a global quantitative assessment of membrane integrity in an sEV preparation (provided that the level of CTB associated CD81 for intact MSC-sEV can be established). In lieu of this, the level of CTB-CD81 is benchmarked against a universally accepted MSC-sEV preparation. CD81+ EVs might also be quantified by imaging flow cytometry, plasmon resonance-based technologies, or by novel, more sensitive fluorescence NTA instruments.

Co-Generation of CMs and MSCs from hiPSCs

Wei et al may be applied (Stem Cell Research, Volume 9, Issue 2, September 2012, Pages 87-100). EVs are created from undifferentiated colonies of hiPSCs and H9 hESCs and differentiated towards cardiomyocytes following a modified cardiac differentiation protocol which involves for example cardiomyogenic medium (CARM) plus a specific p38-MAPK inhibitor SB 203580 (hereby named CARM/SB protocol). After 15 days of cardiac differentiation, 50% of EBs survive and are plated between 15 and 21 days. Both contracting (containing functional cardiomyocytes) and non-contracting EBs give rise to homogenous outgrowth of MSC-like cell population that rapidly migrates from the attached EBs. With the contracting EBs, the beating cell clusters are microdissected out to isolate cardiomyocytes.

Incorporated herein by reference are various ways of making mesenchymal stem cells from hiPSCs; See J Cell Mol Med. 2016 August; 20(8): 1571-1588, Published online 2016 Apr. 21. doi: 10.1111/jcmm.12839 PMCID: PMC4956943, PMID: 27097531, hiPSC-derived iMSCs:

NextGen MSCs as an advanced therapeutically active cell resource for regenerative medicine Vikram Sabapathy and Sanjay Kumar. Additionally and alternatively, we refer to section “Differentiation of hiPSCs to MSCs” of the present patent application. Differentiation of hiPSCs into cells that resemble adult MSCs is an attractive approach to obtain a readily available source of progenitor cells for tissue engineering. Methods typically rely on the addition of soluble factors to affect PSC differentiation.

Preferably, EV characterization is done by a method selected from the group of, i) performing a potency test for the exosomes produced, ii) testing the size distribution of the exosomes produced and iii) testing for an exosomal surface marker.

Methods and devices for characterizing EVs comprise flow cytometry-based analyses (FACS, IFCM, NanoFCM), ELISA, western blotting, or the use of the NanoView platform, an ONI device or the Particle Metrix ZetaView Quatt. “FACS” refers to fluorescence-activated cell sorting. It refers to a method of analyzing and purifying cells based on the presence or absence of specific physical characteristics, such as surface marker expression. Flow cytometers with sorting capabilities detect cells using parameters including cell size, morphology, and protein expression. Thereafter, a droplet-based technology is used to sort cells. IFCM refers to imaging flow cytometry which can be used to determine the concentration of EVs with a diameter smaller than or equal to 400 nm without the need of a prior isolation of EVs from within their occurrence in conditioned medium. In fact, IFCM does not require an EV purification step before or after antibody labelling, thereby avoiding negative impacts on downstream analyses. Most, if not all other technologies require upfront concentration and/or post labelling purification steps. For IFCM analyses, as low as 1 ml of conditioned medium can be used to determine EV content. Also suitable for EV characterization is NanoFCM (Nano-flow cytometry), which has a lower detection limit of 40 nm for exosomes.

Exosome characterization is important. Only certain kinds of exosomes are therapeutically active. Incorporated by reference are various methods as disclosed in Gimona et al. (FEATURED ARTICLE FROM THE ISCT EXOSOMES SCIENTIFIC COMMITTEE|VOLUME 23, ISSUE 5, P373-380, May 1, 2021 (Critical considerations for the development of potency tests for therapeutic applications of mesenchymal stromal cell-derived small extracellular vesicles), Gimona et al., Apr. 9, 2021).

A general approach to the elucidation of the mechanism of action (MoA) of MSC-SEV preparations in eliciting a therapeutic effect is to analyze the RNA or protein cargo for candidates that might mediate the therapeutic effect. To validate the candidate in eliciting the therapeutic effect, a common strategy is to demonstrate a direct cause and effect of a candidate attribute and the therapeutic effect by “knock-out” or “knock-down” experiments. In such experiments, the candidate in the EV is eliminated or reduced by knock-out or knock-down of the gene encoding for the candidate in the EV-producing cells. A direct cause and effect is frequently considered to be validated if the efficacy of the modified EVs is attenuated or lost.

An MSC EV preparation of 80 nm to 1 μm protects against glycerol-induced acute kidney injury; See Bruno et al. (Mesenchymal stem cell-derived microvesicles protect against acute tubular injury, J Am Soc Nephrol. 2009; 20: 1053-1067). One of the associated pathological processes was tissue injury that leads to cell death, and the biological activities induced by this MSC EV preparation were enhanced cell proliferation and apoptosis resistance. The attributes in this preparation that elicited these activities were identified as the messenger RNAs of CCNB1, CDK8 and CDC6; protein growth factors, such as insulin-like growth factor 1 and hepatocyte growth factor; and microRNAs, such as miR-486-5p, miR-126 and miR-34. Therefore, the candidate potency assays to predict the potency of this MSC-EV preparation against acute kidney injury could be quantitative real-time polymerase chain reaction assays to measure the messenger RNA levels of CCNB1, CDK8 and CDC6 or microRNAs, such as miR-486-5p, miR-126 and miR-34. In addition, antibody-based assays, such as enzyme-linked immunosorbent assay, to measure growth factors and proteins in the insulin-like growth factor 1 and hepatocyte growth factor signaling pathways appear appropriate.

Lai et al. (Lai R. C. Exosome secreted by MSC reduces myocardial ischemia/reperfusion injury, Stem Cell Res. 2010; 4: 214-222) indicated that an MSC-sEV preparation of 110-130 nm reduces myocardial ischemia/reperfusion injury by alleviating reperfusion-associated pathological processes such as depleted adenosine triphosphate (ATP) levels, increased oxidative stress and apoptosis by inducing biological activities, such as increasing ATP synthesis, reducing anti-oxidative stress through the degradation of denatured proteins and activating survival signaling via AKT activity. These biological activities could be mapped to ATP-generating enzymes such as pyruvate kinase, 20S proteasome and CD73/ecto-5′-nucleotidase, respectively, present in MSC-sEV preparations. Therefore, probable candidate potency assays to predict the potency of this MSC-sEV preparation against myocardial ischemia/reperfusion injury could be enzymatic assays for pyruvate kinase, 20S proteasome and CD73.

As illustrated by these two examples, potency assays for MSC-sEV preparations are likely to vary for different diseases and may also be different for similar pathological processes (e.g., apoptosis).

As potency assays are one of the gatekeepers for the release of drug products, these assays will have to be stringently validated where possible. According to the ICH guidance for validation of analytical procedures (https://database.ich.org/sites/default/files/Q2_R1_Guideline.pdf), potency assays should be validated on the following key parameters: analytical procedure, which describes the steps to perform the assay in its entirety; specificity, to assess unequivocally the analyte in the presence of other components that may be expected to be present; accuracy, or ability to measure close to the true or reference value; and precision, where measures from multiple sampling of the same sample under the prescribed conditions are in close agreement. Other parameters include detection limit, quantitation limit, linearity range and robustness.

EV cargos can be characterized for example by knockdown/knockout studies, transfection of nucleic acids/proteins/peptides/small molecules into the EVs. For therapeutic approaches, reagents that facilitate uptake of exosomes by cells (such as EV Shuttle and EV Entry Kits) or help in targeting EVs to specific tissues (e.g. XSTAMP), can be employed.

EVs and EV cargos may be analyzed by nanoparticle tracking analyses (NTAs; see e.g. Ludwig et al., 2018; Sokolova et al., 2011) to study EV size and concentration, transmission electron microscopy, RNA-Sequencing, western blotting or proteomics analyses, such as protein arrays or mass spectrometry.

EV cargos can also be analyzed as described in Hansmann et al., which is incorporated herein by reference (Hansmann, G., Chouvarine, P., Diekmann, F. et al. Human umbilical cord mesenchymal stem cell-derived treatment of severe pulmonary arterial hypertension. Nat Cardiovasc Res 1, 568-576 (2022). https://doi.org/10.1038/s44161-022-00083-z).

### Considerations in Designing Potency Assays for MSC-sEV Preparations

MSC-sEV preparations, being biological preparations, impose on the design of potency assays some constraints, such as time. Unlike small molecules, complex biological products such as MSC-sEV preparations may have a much shorter shelf-life that can impose a time constraint on testing for product release. Assays (e.g., animal testing) that require a lengthy period of testing (up to months) may significantly consume the shelf-life of the product and thus reduce commercial viability upon release.

Preferably, the exosomal surface marker is selected from the group of CD63, CD9, CD81, Calnexin, Hsp70 and TSG101.

This may also be done with an ELISA kit. The CD9/CD63 Exosome ELISA Kit is a Sandwich ELISA kit, which utilizes high-performance anti-CD9 and anti-CD63 antibodies. This product detects exosome markers, CD9 and CD63 molecules that are located on the exosome surface in the body fluids or cell culture supernatant; See Cosmobio EXH0102EL. The culture medium of breast cancer cell line, MDA-MB-231, is for example collected, and exosomes are purified by ultracentrifugation. The purified exosomes, 0.781, 1.56, 3.13, 6.25, 12.5, 25, and 50 ng each, are added to the well, and measured using this kit. The Standard curve is created using the measurement of Standard Protein (CD9/CD63 fusion protein) assuming 5 μg of CD9/CD63 fusion protein as 1 unit, the OD450 for 1 unit will be 0.7. For the exosomes purified from MDM-MB-231, the protein concentration for 1 unit of OD450, which will correspond to 0.7, is about 50 ng. Therefore, 50 ng of MDM-MB-231 derived exosomes can be considered as 1 unit of CD9 and CD63 positive exosomes. Presenting the exosome measurement by the units, one can standardize it and/or normalize the measurements, and then will be able to compare exosome measurements directly between different samples or different experiments.

Identification of Exosomes using the tetraspanins CD9, CD63, CD81 and Particle Metrix ZetaView fluorescent Nanoparticle Tracking (f-NTA) may also be done.

More preferably, the exosomal surface marker is CD63 and CD9.

Most preferably, the immortalized clonal cell line of mesenchymal stem cells (IMSC) are characterized by their capability to form fat or bone cells.

Mesenchymal stem cells can be characterized and identified by several ways. One is that they are phenotypically characterized as CD34−, CD105+, and CD90+cells, optionally additionally as CD73+, CD44+, CD45−, CD31−, CD14−, and HLA-DR-. Specified criteria defining the population of MSCs are the following. The condition for the identification of MSCs is the growth of cells in vitro as a population adhering to the substrate, as well as in the case of cells of human origin, a phenotype characterized by the presence of CD44, CD73, CD90, CD105 surface antigens and the lack of expression of proteins such as: CD45, CD31, CD34, CD14, CD11b, CD79a or CD19, TRA 1-60, or class Il histocompatibility complex antigens (HLA II, human leukocyte antigens class II). However, expression of CD105 may vary. In fact, expression of CD105 is known to be influenced by culture conditions, in particular by use of serum (Mark et al., 2013, https://doi.org/10.1155/2013/698076). Furthermore, CD105 expression may in general be lower in iPSC-MSCs as well as in fetal tissue-derived MSCs, potentially suggesting that low CD105 reflects early developmental stages (Spitzhorn et al., 2019, https://doi.org/10.1186/s13287-019-1209-x). Nevertheless, CD105 expression neither appears to correlate with (chondrogenic) differentiation potential of MSCs (Cleary et al., 2016, https://doi.org/10.1016/j.joca.2015.11.018), nor does the absence of CD105 reduce immunomodulatory capacity (Pham et al., 2019, DOI:10.15419/bmrat.v64.538).

The non-MSC, other lineage specific markers comprise CD31 (an endothelial and hematopoietic marker), CD34 (a marker of hematopoietic stem cells and endothelial cells), CD14 (a marker for macrophages), CD45 (a hematopoietic marker), HLA-DR (a marker for macrophages, lymphocytes and monocytes) and TRA 1-60 (a pluripotency marker).

A marker is considered expressed if the measured quantity or the detected fluorescence is above the measured quantity or background fluorescence intensity detected with a control or unspecific antibody, e.g. an lgG control. For example, if a MSC-specific marker, such as CD44 is analyzed, the marker is considered expressed if a cell shows fluorescence intensity above the background level detected by an IgG control. Ideally, all the analyzed cells or at least 90% or at least 80% express the marker.

Moreover, the cells must have the ability to differentiate towards osteoblasts, adipocytes and chondroblasts.

In addition to the markers mentioned in the ISCT guidelines, the following antigens may be used: STRO-1 (antigen of the bone marrow stromal-1 antigen, cell surface antigen expressed by stromal elements in human bone marrow-1), VCAM/CD106 (vascular cell adhesion molecule 1) and MCAM/CD146 (melanoma cell adhesion molecule), which characterizes cells growing in vitro in an adherent form, with a high degree of clonogenicity and multidirectional differentiation ability.

The invention also relates to a stem cell line produced according to the invention.

The invention relates to the cell line according to the invention, for use as a medicament.

The invention relates to the cell line according to the invention for use in treating a disease that is amenable to treatment with stem cell therapy.

These are preferably selected from the group of bone marrow, skin, heart, and corneal transplantation, graft versus host disease, hepatic and renal failure, lung injury, bronchopulmonary dysplasia, rheumatoid arthritis, treatment of autoimmune diseases such as Crohn's disease, ulcerative colitis, multiple sclerosis, lupus and diabetes; prevention of allograft rejection, neurological disorders and cardiovascular medicine; as well as Acute lymphoblastic leukemia (ALL), Acute myeloid leukemia (AML), Burkitt's lymphoma, Chronic myeloid leukemia (CML), Juvenile myelomonocytic leukemia (JMML), (Non-Hodgkin's lymphoma Hodgkin's lymphoma, Lymphomatoid granulomatosis, Myelodysplastic syndrome (MDS), Chronic myelomonocytic leukemia (CMML), Bone Marrow Failure Syndromes, Amegakaryocytic thrombocytopenia, Autoimmune neutropenia (severe), Congenital dyserythropoietic anemia, Cyclic neutropenia, Diamond-Blackfan anemia, Evan's syndrome, Fanconi anemia, Glanzmann's disease, Juvenile dermatomyositis, Kostmann's syndrome, Red cell aplasia, Schwachman syndrome, Severe aplastic anemia, Congenital sideroblastic anemia, Thrombocytopenia with absent radius (TAR syndrome), Dyskeratosis congenital, Blood Disorders, Sickle-cell anemia (hemoglobin SS), HbSC disease, Sickle βo Thalassemia, α-thalassemia major (hydrops fetalis), β-thalassemia major (Cooley's anemia), β-thalassemia intermedia, E-βo thalassemia, E-βo thalassemia, Metabolic Disorders, Adrenoleukodystrophy Gaucher's disease (infantile), Metachromatic leukodystrophy, Krabbe disease (globoid cell leukodystrophy), Gunther disease, Hermansky-Pudlak syndrome, Hurler syndrome, Hurler-Scheie syndrome, Hunter syndrome, Sanfilippo syndrome, Maroteaux-Lamy syndrome, Mucolipidosis Type II, III, Alpha mannosidosis, Niemann Pick Syndrome, type A and B, Sandhoff Syndrome, Acute liver failure, Tay-Sachs Disease, Batten disease (inherited neuronal ceroid lipofuscinosis), Lesch-Nyhan disease, Immunodeficiencies, Ataxia telangiectasia, Chronic granulomatous disease, DiGeorge syndrome, IKK gamma deficiency, Immune dysregulation polyendocrineopathy, X-linked Mucolipidosis, Type II, Myelokathexis X-linked immunodeficiency, Severe combined immunodeficiency, Adenosine deaminase deficiency, Wiskott-Aldrich syndrome, X-linked agammaglobulinemia, X-linked lymphoproliferative disease, Omenn's syndrome, Reticular dysplasia, Thymic dysplasia, Leukocyte adhesion deficiency, Other Osteopetrosis, Langerhans cell histiocytosis, Hemophagocytic lymphohistiocytosis, Acute & Chronic Kidney Disease, Acute Kidney failure, Alzheimer's disease, Anti-Aging, Arthritis, Asthma, Cardiac Stem Cell Therapy, Cerebral Infarction (Stroke), Cerebral Palsy (Stroke), Chronic Obstructive Pulmonary Disease (COPD), Congestive Heart Failure, Diabetes Mellitus (Type I & II), Fibromyalgia, Immune Deficiencies, Ischemic Heart Disease, Lupus, Multiple Sclerosis, Myocardial/Cardiac Infarction, Heart failure, Osteoarthritis, Osteoporosis, Parkinson's Disease, Peripheral Arterial Disease, Rheumatoid Arthritis, Stem Cell Therapy in Plastic Surgery, Traumatic Brain Injury, Neurological Diseases, pulmonary hypertension, pulmonary arterial hypertension, various forms of brain damage and spinal cord injury in newborns, children, and adults such as, but not limited to, white matter brain damage, periventricular leukomalacia, intracranial hemorrhage, cerebral hemorrhage, arterial stroke, cerebral venous thrombosis, traumatic brain injury (TBI), brain concussion, spinal cord injury (SCI), neonatal encephalopathy, cerebral palsy, infectious brain damage, encephalitis, meningitis, amyotrophic lateral sclerosis (ALS), Alzheimer dementia, Parkinson, and Multiple sclerosis.

The invention also relates to the use of a stem cell line according to the invention for producing EVs.

The invention also relates to a method of producing mesenchymal stem cell derived exosomes comprising the steps of,
i) performing a method of producing a clonal mesenchymal stem cell line capable of producing exosomes (MSC-derived extracellular vesicles) comprising the steps of,
a. providing human induced pluripotent stem cells (hiPSCs),
b. generating therefrom an immortalized clonal cell line of mesenchymal stem cells (IMSCs),
c. characterizing the potential of the IMSCs to produce exosomes,
ii) cultivating the desired IMSCs such that the IMSCs produce exosomes,
iii) isolating the exosomes.

Preferably, the exosome have a density as determined by density gradient centrifugation of 1.13 per ml to 1.19 g per mL.

Preferably, here the hiPSCs have a homozygous leukocyte antigen (HLA) haplotype (HLA-homo HP) and wherein the hiPSCs were generated by means of one or more Yamanaka factors such as but not limited to Myc, Oct3, Oct4, Sox2 and Klf4 and stem from preferably umbilical cord blood tissue.

It is preferred if method encompasses transfecting an umbilical cord blood stem cell with a nucleic acid encoding an OCT4 protein and a nucleic acid encoding a SOX2 protein to form a transfected cord blood stem cell and allowing said transfected cord blood stem cell to divide thereby forming said induced pluripotent stem cell.

Preferably, the generation of immortalized clonal cell line of mesenchymal stem cells (IMSC) is done by means of a tissue specific media, such as a cardiomyogenic medium (CARM) and optionally a specific p38-MAPK inhibitor, wherein after generation the resulting IMSC expresses one or more of the following markers selected from the group of CD29, CD44, CD73, CD90, and CD105.

Ideally, the characterization is done by a method selected from the group of, i) performing a potency test for the exosomes produced, ii) testing the size distribution of the exosomes produced and iii) testing for a exosomal surface marker.

Multi-Donor Mixed Lymphocyte Reaction (mdMLR) Assay

The mdMLR is a functional assay allowing for an evaluation of the immunomodulatory potential/activity of MSC-EVs and may include testing of different individual MSC-EV preparations. Details on the mdMLR assay are described in example 10.

The invention relates to a method of testing the immunomodulatory activity of extracellular vesicles obtained from human induced pluripotent stem cell-derived mesenchymal stem cells (hiPSC-derived MSC-EVs) in a multi-donor mixed lymphocyte reaction (mdMLR) assay, the method comprising:
a) obtaining peripheral blood mononuclear cells (PBMCs);
b) obtaining hiPSC-derived MSC-EVs;
c) culturing said PBMCs

in the presence of said hiPSC-derived MSC-EVs (EV-treated PBMCs),

in the absence of said hiPSC-derived MSC-EVs (EV-untreated PBMCs);
a) measuring

the number of activated CD4+-and/or CD8+-T cells from step c)i), and

the number of activated CD4+-and/or CD8+-T cells from step c)ii);
a) calculating a fold change as defined by the number of activated CD4+-and/or CD8+-T cells of step d)i) over the number of activated CD4+-and/or CD8+-T cells of step d)ii),

wherein the hiPSC-derived MSC-EVs are considered to possess immunomodulatory activity, if the fold change is lower than 0.5.

The fold change is calculated by the number of activated CD4+-and/or CD8+-T cells of EV-treated PBMCs divided by the number of activated CD4+-and/or CD8+-T cells of EV-untreated PBMCs.

Alternatively, the hiPSC-derived MSC-EVs are considered to possess immunomodulatory activity, if the fold change is lower than 1, or lower than 0.9, or lower than 0.8, or lower than 0.7, or lower than 0.6, or lower than 0.4, or lower than 0.3.

“EV-untreated PBMCs” does not necessarily only refer to PBMCs to which no EVs were added, but can also comprise PBMCs treated with other EVs serving as controls within the assay. Examples therefore are hPL-derived EVs (EVs prepared from fresh, hPL-supplemented culture medium) or known non-active EVs (which may also be obtained from MSCs derived from other sources than hiPSCs). Furthermore, positive controls can also be included in the assay, for example known active EVs (which may also be obtained from MSCs derived from other sources than hiPSCs).

“PBMCs” are human peripheral blood mononuclear cells. These are mononuclear (with a round nucleus) immune cells which originate in the bone marrow and are secreted into peripheral circulation. They are involved in humoral and cell-mediated immunity. PBMCs comprise T cells, B cells, natural killer (NK) cells and monocytes.

Preferably, the PBMCs are derived from buffy coats of healthy peripheral blood donors.

Buffy coat is a blood component obtained by centrifugation of a whole blood unit/sample, which contains the majority of leukocytes and platelets (the latter depending on the centrifugation).

More preferably, the PBMCs are thawed from a previously pooled, aliquoted and cryopreserved PBMC stock prior to use.

Preferably, 200.000 PBMC cells are used for the mdMLR assay. Alternatively, 100.000 cells or 150.000 cells or 250.000 cells Or 300.000 cells or between 100.000 cells and 300.000 cells can be used.

In one embodiment, the mesenchymal stem cell-derived extracellular vesicles (MSC-EVs) are collected using conditioned medium obtained from hiPSC-derived MSCs.

Preferably, the MSC-EVs were collected using conditioned medium obtained from hiPSC-derived MSCs, wherein the conditioned medium was centrifuged (e.g. for 10 min at 2.000×g) to remove cells from the conditioned medium (CM) prior to use in the mdMLR assay. CM can be used freshly or from a frozen aliquot or stock. If frozen aliquots are used, it is preferred to include a centrifugation step (e.g. 10 min at 2.000×g) to remove cells prior to freezing.

In one embodiment, the activated CD4+-and/or CD8+-T cells are considered activated if they express CD25 and CD54.

Preferably, measuring of the number of activated CD4+-and/or CD8+-T cells is performed by using at least one of the following methods: fluorescence-activated cell sorting (FACS), imaging flow cytometry (IFCM), western blotting, or enzyme-linked immunosorbent assay (ELISA).

Therapeutic efficacy depends on the immunomodulatory activity/potential of each therapeutically applied EV preparation. The mdMLR assay, optionally in combination with several methods that can be used to analyze EV cargos, enables the identification and distinguishability of active vs. inactive EVs, a critical step towards obtaining therapeutically active EVs over and over again. In order to further improve therapeutic potential, the invention relates to the use of hiPSC-derived MSCs, which have the potential to produce more EVs than bone marrow-derived EVs (FIG. 14), the latter already successfully having been used in therapy (incorporated herein by reference are
1.) Doeppner T R, Herz J, Görgens A, Schlechter J, Ludwig A K, Radtke S, de Miroschedji K, Horn PA, Giebel B, Hermann D M. Extracellular Vesicles Improve Post-Stroke Neuroregeneration and Prevent Postischemic Immunosuppression. Stem Cells Transl Med. 2015 October;4(10):1131-43. doi: 10.5966/sctm.2015-0078. Epub 2015 Sep. 3. PMID: 26339036; PMCID: PMC4572905 .;
2.) Karla Drommelschmidt, Meray Serdar, Ivo Bendix, Josephine Herz, Frederik Bertling, Sebastian Prager, Matthias Keller, Anna-Kristin Ludwig, Vikas Duhan, Stefan Radtke, Kyra de Miroschedji, Peter A. Horn, Yohan van de Looij, Bernd Giebel, Ursula Felderhoff-Müser, Mesenchymal stem cell-derived extracellular vesicles ameliorate inflammation-induced preterm brain injury, Brain, Behavior, and Immunity, Volume 60, 2017, Pages 220-232, ISSN 0889-1591, https://doi.org/10.1016/j.bbi.2016.11.011.;
3.) Giebel B, Kordelas L, Börger V. Clinical potential of mesenchymal stem/stromal cell-derived extracellular vesicles. Stem Cell Investig. 2017 Oct. 24;4:84. doi: 10.21037/sci.2017.09.06. PMID: 29167805; PMCID: PMC5676188.).

Furthermore, in order to reduce heterogeneity or variability in the production process, the MSCs can be cultured in serum-free medium with defined components (as demonstrated e.g. in FIGS. 4B, 8 and 9), thereby enabling standardized and highly reproducibly production methods and avoiding batch-to batch variances which may otherwise occur due to varying serum compositions.

Preferably, the exosomal surface marker is selected from the group of CD 63, CD 9, Calnexin, Hsp70 and TSG101, more preferably, the exosomal surface marker is CD 63 and CD 9.

Preferably, the immortalized clonal cell line of mesenchymal stem cells (IMSC) is characterized by their capability to form fat or bone cells.

The invention also relates to an EV produced according to the method of the invention. It relates to the use of the EV as a medicament.

Preferably, the disease is selected from the group of bone marrow, skin, heart, and corneal transplantation, graft versus host disease, hepatic and renal failure, lung injury, bronchopulmonary dysplasia, rheumatoid arthritis, treatment of autoimmune diseases such as Crohn's disease, ulcerative colitis, multiple sclerosis, lupus and diabetes; prevention of allograft rejection, neurological disorders and cardiovascular medicine; as well as Acute lymphoblastic leukemia (ALL), Acute myeloid leukemia (AML), Burkitt's lymphoma, Chronic myeloid leukemia (CML), Juvenile myelomonocytic leukemia (JMML), Non-Hodgkin's lymphoma Hodgkin's lymphoma, Lymphomatoid granulomatosis, Myelodysplastic syndrome (MDS), Chronic myelomonocytic leukemia (CMML), Bone Marrow Failure Syndromes, Amegakaryocytic thrombocytopenia, Autoimmune neutropenia (severe), Congenital dyserythropoietic anemia, Cyclic neutropenia, Diamond-Blackfan anemia, Evan's syndrome, Fanconi anemia, Glanzmann's disease, Juvenile dermatomyositis, Kostmann's syndrome, Red cell aplasia, Schwachman syndrome, Severe aplastic anemia, Congenital sideroblastic anemia, Thrombocytopenia with absent radius (TAR syndrome), Dyskeratosis congenital, Blood Disorders, Sickle-cell anemia (hemoglobin SS), HbSC disease, Sickle βo Thalassemia, α-thalassemia major (hydrops fetalis), β-thalassemia major (Cooley's anemia), β-thalassemia intermedia, E-βo thalassemia, E-β+ thalassemia, Metabolic Disorders, Adrenoleukodystrophy Gaucher's disease (infantile), Metachromatic leukodystrophy, Krabbe disease (globoid cell leukodystrophy), Gunther disease, Hermansky-Pudlak syndrome, Hurler syndrome, Hurler-Scheie syndrome, Hunter syndrome, Sanfilippo syndrome, Maroteaux-Lamy syndrome, Mucolipidosis Type II, III, Alpha mannosidosis, Niemann Pick Syndrome, type A and B, Sandhoff Syndrome, Acute liver failure, Tay-Sachs Disease, Batten disease (inherited neuronal ceroid lipofuscinosis), Lesch-Nyhan disease, Immunodeficiencies, Ataxia telangiectasia, Chronic granulomatous disease, DiGeorge syndrome, IKK gamma deficiency, Immune dysregulation polyendocrineopathy, X-linked Mucolipidosis, Type II, Myelokathexis X-linked immunodeficiency, Severe combined immunodeficiency, Adenosine deaminase deficiency, Wiskott-Aldrich syndrome, X-linked agammaglobulinemia, X-linked lymphoproliferative disease, Omenn's syndrome, Reticular dysplasia, Thymic dysplasia, Leukocyte adhesion deficiency, Other Osteopetrosis, Langerhans cell histiocytosis, Hemophagocytic lymphohistiocytosis, Acute & Chronic Kidney Disease, Acute Kidney failure, Alzheimer's disease, Anti-Aging, Arthritis, Asthma, Cardiac Stem Cell Therapy, Cerebral Infarction (Stroke), Cerebral Palsy (Stroke), Chronic Obstructive Pulmonary Disease (COPD), Congestive Heart Failure, Diabetes Mellitus (Type I & II), Fibromyalgia, Immune Deficiencies, Ischemic Heart Disease, Lupus, Multiple Sclerosis, Myocardial/Cardiac Infarction, Heart failure, Osteoarthritis, Osteoporosis, Parkinson's Disease, Peripheral Arterial Disease, Rheumatoid Arthritis, Stem Cell Therapy in Plastic Surgery, Traumatic Brain Injury, Neurological Diseases, pulmonary hypertension, pulmonary arterial hypertension, various forms of brain damage and spinal cord injury in newborns, children, and adults such as, but not limited to, white matter brain damage, periventricular leukomalacia, intracranial hemorrhage, cerebral hemorrhage, arterial stroke, cerebral venous thrombosis, traumatic brain injury (TBI), brain concussion, spinal cord injury (SCI), neonatal encephalopathy, cerebral palsy, infectious brain damage, encephalitis, meningitis, amyotrophic lateral sclerosis (ALS), Alzheimer dementia, Parkinson, and Multiple sclerosis.

The invention also relates to a pharmaceutical composition comprising an EV according to the invention.

### EXAMPLES

Methods can be done as disclosed in WO 2010/148334.

Umbilical cord blood (CB) samples are obtained. Mononuclear cells (MNC) are isolated from CB using Lympholyte-H (Cederlane, Ontario, CA) density gradient centrifugation. CD 133+ cells are positively selected using Mini-Macs immunomagnetic separation system (Miltenyi Biotec, Bergisch Gladbach, Germany). Purification efficiency is verified by flow cytometric analysis staining with 10 CD133-phycoerythrin (PE; Miltenyi Biotec, Bergisch Gladbach, Germany) antibody.

### Example 1

### Constructs and Retroviral Production

OCT4 and SOX2 human cDNAs are amplified from ES total RNA by RT-PCR; human KLF4 is amplified from IMAGE clone 5111134 and the mutant human cMYCT58A is amplified from a DNA template. cDNAs are cloned into the EcoRI/Clal sites of a modified pMSCVpuro vector that allows the expression of N-terminal FLAG-tagged proteins. pMXs-OSKMG is constructed as follows: the mouse Oct4 cDNA is amplified using a reverse primer eliminating the Oct4 stop codon and adding a BspEl site and cloned into pCRII (Invitrogen) to give pCRII-Oct4-Bsp (oriented NotI-5′cDNA3′-Acc651). The mouse Sox2 cDNA is amplified using a forward primer containing an AgeI site followed by P2A peptide sequence and a reverse primer eliminating the Sox2 stop codon and containing a BspEI site; this fragment is cloned in pCRII to give pCRII-Age-Sox2-Bsp (oriented NotI-5′cDNA3′-Acc651). pCRII-Age-Sox2-Bsp is cut by AgeI and Acc65I and cloned into pCRII-Oct4-Bsp cut BspEI-Acc651 producing pCRII-Oct4-P2A-Sox2-BspEI. The same cloning approach is repeated twice in order to incorporate mouse Klf4 and eGFP (producing pCRII-OSKG) or mouse Klf4, c-Myc and eGFP (producing pCRII-OSKMG). Finally pCRII-OSKG and pCRIIOSKMG is cut EcoRI and cloned into a unique EcoRI site of the retroviral empty vector pMXs, producing pMXs-OSKG and pMXs-OSKMG. Retroviruses for the four factors are independently produced after transfecting the cell line Phoenix Amphotropic using Fugene 6 reagent (Roche) according to manufacturer's directions.

After 24 hours, the medium was replaced, cells were incubated at 32° C., and viral supernatant is harvested every 12 hours.

### Example 2

### Transduction of CD133+ Cells

CB CD133+ cells (1×10⁵ cells per ml) are pre-stimulated for 24 h in DMEM supplemented with 10% of FBS in the presence of SCF (50 ng/ml)+Flt3 (50 ng/ml)+TPO (10 ng/ml)+IL-6 (10 ng/ml) (PeproTech). Multi-well non-tissue culture-treated plates are coated with retronectin (Takara, Otsu, Japan, www.takara-bio.com), a fibronectin fragment CH-296 (15 mg/cm²), and preloaded by centrifuging the plates with a filtered 1:1:1:1 mix of retroviral supernatant for OCT4, SOX2, KLF4, and c-MYC factors at the 2,500 RPM for 30 minutes.

About 80,000 CD133+ cells are plated in the presence of DMEM +10% FBS and the cytokine cocktail mentioned above. Every 12 h, half of the medium is replaced with fresh viral supernatant containing the cytokine cocktail and incubated at 37° C., 5% CO₂; three infection cycles were performed.

At day 3, the cells are harvested and transferred into 6 well-plates containing irradiated human fibroblasts and ES medium, consisting of KODMEM medium (Invitrogen) supplemented with 20% KO-Serum Replacement (GIBCO), non-essential amino acids (Lonza), 2-mercaptoethanol (GIBCO), Penicillin/Streptomycin (GIBCO), GlutaMAX™ (Invitrogene), and 10 ng/ml bFGF (Peprotech). CBiPS cells are cultured on top of irradiated human fibroblasts and picked mechanically.

### Example 3

### Purification of Total RNA and Quantitative RT-PCR

Isolation of total RNA from CB CD133+ stem cells, hES cells, KiPS cells and CBiPS is performed using either Trizol® reagent (Invitrogen, Carlsbad, CA) or RNAqueous®-Micro kit (Ambion Inc., Austin TX) based on the cell number available. All samples are treated with TURBO DNase inhibitor (Ambion) to remove any residual genomic DNA and 1 μg of RNA was used to synthesize cDNA using the Invitrogen SuperScript™ II Reverse Transcriptase kit. 25 ng of cDNA are used to quantify gene expression by Quantitative RT-PCR using primers as previously described.

### Example 4

### GeneChip® Expression Analysis.

The GeneChip® microarray processing is performed by the Functional Genomica Core in the Institute for Research in Biomedicine (Barcelona, Spain) according to the manufacturer's protocols (Affymetrix, Santa Clara, CA). The amplification and labelling is processed as indicated in Nugen protocol with 25 ng starting RNA. For each sample, 3.75 μg ssDNA were labelled and hybridized to the Affymetrix HG-U133 Plus 2.0 chips. Expression signals are scanned on an Affymetrix GeneChip Scanner (7G upgrade). The data extraction is done by the Affymetrix GCOS software v.1.4.

The statistical analysis of the data was performed using the program R from the R Project for Statistical Computing. Raw data is normalized using the gcRMA algorithm implemented in R, and a hierarchical clustering using Pearson correlation coefficients is performed on the normalized data.

### Example 5

Maintenance of hiPSCs

hiPSCs are cultured in StemMACS iPS-Brew XF medium (XF medium; Miltenyi Biotec), optionally supplemented with 1:100 Penicillin-Streptomycin (PS; Thermo Fisher Scientific) and optionally supplemented with 10 μM final concentration of Rock inhibitor (Y-27632 (Y); R&D Systems). This prepared medium is used up to two weeks. The cells are passaged every 3-4 days at a density of 200.000-250.000 cells per well of a 6-well plate. Of note, the wells were pre-coated for at least 1 hour at 37° C. using 2 ml XF medium+PS+Y, supplemented with 3 μl iMatrix-511. Cell passaging is performed by washing the cells with 2 ml of phosphate-buffered saline (PBS), followed by using 1 ml of Accutase (Sigma), supplemented with 10 μM of Rock inhibitor (Y-27632; R&D Systems), per well, until cells detach from the bottom of the wells. The same volume used for Accutase+Y treatment, is then used in terms of pre-warmed XF medium+PS+Y. Cells are centrifuged at 300xg for 3 min, supernatant is removed and cells are resuspended in 2 ml of pre-warmed XF medium+PS+Y per harvested well. 10 μl are used for counting and 200.000-250.000 cells (100.000 if used for subsequent “MSC-differentiation” experiments) are seeded per well into each pre-coated well of a 6-well plate. The plate is slowly moved in an infinity symbol pattern in order to evenly distribute the cells and placed into a CO₂ incubator. The medium is replaced by fresh pre-warmed XF medium+PS every 1-3 days. The hiPSCs should be 50-100% confluent and undifferentiated, before passaging or seeding for an experiment.

The skilled artisan will appreciate, however, that other ways of passaging cells (or hiPSCs), which are known in the art, may also be suitable and the above-mentioned example of hiPSC maintenance shall not be viewed as limiting the scope of the invention.

### Example 6

### Example 6.1: In vitro Differentiation

EBs formation is induced from colony fragments mechanically collected and then maintained in suspension in presence of ES medium for 24 hours. A pre-condition culture is done for 2-3 days where the EBs were maintained in the three different differentiation medium onto ultra-low attachment plates. In particular, for endoderm differentiation the EBs are cultured in the presence of KO-DMEM medium supplemented with 10%fetal bovine serum (FBS), 2 mM L-glutamine, 0.1 mM 2-mercaptoethanol, non-essential amino acids, and penicillin-streptomycin. For mesoderm differentiation, the same medium described above is used, but adding ascorbic acid (0.5 mM). For ectoderm induction, the EBs are cultured in N2/B27 medium. After the precondition step, the EBs in endoderm and mesoderm conditions are transferred to 0.1% gelatine-coated plastic chamber slides and cultured in differentiation medium and differentiation medium plus acid ascorbic (0.5 nM) respectively, for 2 weeks. For the ectoderm differentiation, the EBs are transferred onto stromal cell line PA6 and in the presence of N2/B27 medium for 2 weeks. The medium for each condition is changed every other day.

### Example 6.2: Differentiation of hiPSCs to MSCs

Differentiation of hiPSCs to MSC cells (MSCs) can be achieved via WNT activation. Non-limiting example protocols for such a differentiation method are disclosed in more detail hereinafter.

### Example 6.2.1

The preferred method of differentiation of hiPSCs to MSCs is described hereinafter. hiPSCs are seeded at a density of 100.000 cells per pre-coated well of a 6-well plate as described above in Example 5. The next day (day 0), an even distribution of the cells (including the formation of only loose colonies) and a flat morphology are confirmed. Cells are washed once with PBS, before 4 ml of MSCI medium (XF medium+4 μM CHIR99021 (CHIR; STEMCELL Technologies)+1:100 PS) is added. The MSCI medium is used for a total of 6 days and is replaced every 1-3 days. Of note, the XF medium with PS added (XF+PS) can be stored at 2-8° C. up to 1 week. However, CHIR99021 is added freshly each time prior to replacing the medium of the cells.

On day 5, the medium comprising already some floating MSC progenitors is collected (1 ml of pre-warmed MSCI medium is added to the remaining cells in the well) and centrifuged at 300×g for 1 min. Cells are resuspended in an adequate volume (volume=2×number of wells from which medium was collected) of pre-warmed MSCI medium, while trying to preserve the clumps. 2 ml of resuspended cells are added to each well. The procedure of day 5 is repeated on day 6, although MSCG medium (StemMacs MSC Expansion Media (Miltenyi Biotec)+1:100 PS+700 μl StemMacs MSC Expansion Media Supplement (Miltenyi Biotec)) is used instead of MSCI medium. The MSCG medium is used in order to further differentiate the hiPSC-derived MSC progenitors from day 6 onwards. On day 7, the medium is changed and from that day onwards, every two to three days, the medium is replaced with fresh MSCG medium. MSCG medium is also used to further passage and maintain the hiPSC-derived MSCs. The MSCG medium is stored at 2-8° C. up to 1 week.

Once the cells are more than 90% confluent, they are passaged at 1:2 ratio on Laminin-coated 6-well plates (Laminin: 3 μl iMatrix-511 in 2 ml of MSCG medium) (P1). From the next passage (P2) onwards, the hiPSC-induced MSCs are seeded on plastic. A passaging ratio of 1:2 is maintained for 3 passages. From passage 4 onwards, the cells can be diluted at 200.000 cells per well of a 6-well plate with 2 ml MSCG medium. Medium is replaced the next day after seeding and then every 2 days. Every Friday, the MSCs are treated with 3-4 ml MSCG medium.

### Example 6.2.2

To assess the influence of hiPSC density on differentiation, the hiPSC line R26-6 was seeded at different cell densities (2.5K, 5K, 10K, 20K, 40K cells/cm²) in 6 well-plates with XF medium, supplemented with penicillin/streptomycin (PS)). The following day, the cells were treated with XF medium+PS+4 μM CHIR99021 for 6 days. Then the medium was replaced with low glucose MSC medium with 10% FCS. From the second day of passage 2 onwards, the MSCs were grown in low glucose, 10% human Platelet Lysate (hPL) medium. A fibroblastoid morphology of the differentiated cells could be observed. Both MSC-associated features (fibroblastoid morphology and adherence to plastic/Laminin-coated plates) were observed irrespective of the cell density of hiPSCs at the beginning of the differentiation of the hiPSCs.

### Example 6.3: Maintenance of Induced Mesenchymal Stromal/Stem Cells (MSCs)

A non-limiting example of passaging and maintaining an MSC culture is described hereinafter. MSCs are treated with 1 ml TrypLE (Thermo Fisher Scientific) per well of a 6-well plate to detach the cells from the plate. Cells are pipetted up and down to bring them into solution. 3 ml of MSCG medium is added and the cell suspension is collected and centrifuged at 500×g for 3 min. The supernatant is discarded and the cells are resuspended in MSC medium. The cells are seeded onto plates or into flasks at a 1:2 ratio (until the 3″ passage) or at 20.000 cells/cm² (from passage 4 onwards).

The skilled artisan will appreciate, however, that other ways of passaging the cells, which are known in the art, may also be suitable and the above-mentioned example of MSC maintenance shall not be viewed as limiting the scope of the invention.

### Example 6.4: Analysis of Surface Markers on the Hipsc-Derived MSCs Using Flow Cytometry

A non-limiting example of a flow cytometry analysis of the induced MSCs is described hereinafter. Cells are washed with PBS prior to the addition of 1 ml of accutase. Upon accutase treatment, the plates are placed in the CO₂ incubator for 4.0 to 4.5 minutes. Neutralization of accutase is performed by using 1 ml of MSCG medium. Cells were then transferred into a 15 ml tube and centrifuged at 300×g for 2-3 min. The supernatant is discarded and 1 ml of PBS is added. The cells are vortexed shortly and centrifuged at 300×g for 2 min. The supernatant is discarded and cells are resuspended in PBS at a concentration of 200.000-1.000.000 cells/100 μl PBS per antibody and 100 μl are transferred into several 1.5 ml tubes. Depending on the concentration of the antibody, 1-5 μl of each antibody are added to the 100 μl of cells for staining. Each tube is vortexed shortly for 5 sec and incubated for 20 min at room temperature or 37″° C. in the dark. 300 μl of PBS are added and the stained cells are centrifuged at 400×g for 1 min. The supernatant is discarded and the cells are resuspended in PBS:

if the primary antibodies are pre-stained, 300 μl of PBS are added to each tube and the cells are resuspend by vortexing shortly for 5 sec prior to performing flow cytometry;

If the primary antibodies are not pre-stained, 100 μl of PBS are added, as well as an adequate volume of the secondary antibody (final concentration according to the manufacturer's instructions). Then, the tubes are shortly vortexed for 5 seconds and incubated for 20 minutes. After incubation, 300 μl of PBS are added and the cells are centrifuged at 400×g for 1 min. The supernatant is discarded, 300 μl of PBS is added to each tube and the cells are resuspend by vortexing shortly for 5 sec prior to performing flow cytometry.

After detection is completed, the flow cytometry results are analyzed using a suitable software, such as, but not limited to, FlowJo. Suitable flow cytometry softwares and analysis methods are known by those skilled in the art and shall all be encompassed within the methods of this invention.

The skilled artisan will appreciate, however, that other ways of preparing cells for a flow cytometric analysis, which are known in the art, may also be suitable and the above-mentioned example shall not be viewed as limiting the scope of the invention. Other protocols may thus also be used in order to prepare and analyze cells for and by flow cytometry.

The antibodies used for flow cytometry are listed below:

| | | | Catalogue | |
|---|---|---|---|---|
| Antibody | | Company | number | Preparation |
| MSC marker | α-CD44 G-44-26 | BD Bioscience | 555476 | Stored at 2-8° C. or, |
| antibodies | | | | alternatively, at −20° C. |
| | | | | after addition of 80% |
| | | | | (v/v) glycerol to 10% |
| | | | | final concentration |
| | α-CD73 antibody | BD Biosciences | BUV661 | Stored at 2-8° C. or, |
| | | | | alternatively, at −20° C. |
| | | | | after addition of 80% |
| | | | | (v/v) glycerol to 10% |
| | | | | final concentration |
| | α-CD90 (Thy1) | BioLegend | 328135 | Stored at 2-8° C. or, |
| | antibody | | | alternatively, at −20° C. |
| | | | | after addition of 80% |
| | | | | (v/v) glycerol to 10% |
| | | | | final concentration |
| | α-CD105 | BioLegend | 323202 | Stored at 2-8° C. or, |
| | antibody | | | alternatively, at −20° C. |
| | | | | after addition of 80% |
| | | | | (v/v) glycerol to 10% |
| | | | | final concentration |
| Other lineage- | α-CD14 MEM-15 | Thermo Fisher | MA119223 | Stored at 2-8° C. or, |
| specific | antibody | Scientific | | alternatively, at −20° C. |
| marker (non- | | | | after addition of 80% |
| MSC marker) | | | | (v/v) glycerol to 10% |
| antibodies | | | | final concentration |
| | α-CD31 (PECAM- | BioLegend | 303104 | Stored at 2-8° C. or, |
| | 1) antibody | | (FITC- | alternatively, at −20° C. |
| | | | labelled) | after addition of 80% |
| | | | | (v/v) glycerol to 10% |
| | | | | final concentration |
| | α-CD34 antibody | BD Bioscience | 555821 | Stored at 2-8° C. or, |
| | | | (FITC- | alternatively, at −20° C. |
| | | | labelled) | after addition of 80% |
| | | | | (v/v) glycerol to 10% |
| | | | | final concentration |
| | α-CD45 antibody | Miltenyi | 130-113- | Stored at 2-8° C. or, |
| | | Biotec | 118 (FITC- | alternatively, at −20° C. |
| | | | labelled) | after addition of 80% |
| | | | | (v/v) glycerol to 10% |
| | | | | final concentration |
| | α-HLA-DR | BD Bioscience | 550853 | |
| | antibody | | (FITC- | |
| | | | labelled) | |
| | α-TRA 1-60 | | | |
| | antibody | | | |
| Control | α-IgG antibody | | | |
| antibody | | | | |

The skilled artisan will appreciate, that any other antibody suitable for flow cytometry analyses and recognizing the target of interest, can be used for the methods of the invention.

### Example 6.5: Differentiation of MSCs

### Example 6.5.1: Osteogenic Differentiation

The iPSC-derived MSCs are seeded in a 12-well plate at 10.000 cells/cm² in StemPro MSC SFM XenoFree MSC expansion medium (50 ml StemPro MSC SFM XenoFree medium (Thermo Fisher Scientific)+500 μl StemPro MSC SFM XenoFree Supplement (Thermo Fisher Scientific)+500 μl PS) and incubated overnight in a CO₂ incubator. The next day, the medium is replaced with the pre-warmed StemPro Osteogenesis medium (22.25 ml StemPro® Osteocyte/Chondrocyte Differentiation Basal Medium (Gibco)+2.5 ml StemPro Osteogenesis supplement (Gibco)+250 μl PS) and the incubation is continued in the CO₂ incubator. MSCs expand as they differentiate in the Osteogenesis medium. Medium is replaced with fresh pre-warmed Osteogenesis medium every 3-4 days. On day 21 or later, osteogenesis can be analyzed using the Alizarin Red S stain analysis.

### Alizarin Red S Stain Analysis

The medium is removed from the wells of the 12-well plate. Cells are rinsed once with PBS (1 ml). PBS is removed from the wells and cells are fixed with 4% formaldehyde solution (diluted in PBS) for 30 minutes. After fixation, the cells are rinsed twice with distilled water and stained with 2% Alizarin Red S solution for 2-3 minutes on a plate shaker at room temperature. The cells are then rinsed 3-4 times with 2 ml deionized water, visualized under the light microscope and images are taken.

### Example 6.5.2: Adipogenic Differentiation

iPSC-derived MSCs are seeded in a 12-well plate at 80.000 cells/cm² in StemPro MSC SFM XenoFree MSC expansion medium (50 ml StemPro MSC SFM XenoFree medium (Thermo Fisher Scientific)+500 μl StemPro MSC SFM XenoFree Supplement (Thermo Fisher Scientific)+500 μl PS) and incubated overnight in a CO₂ incubator. The next day, the medium is replaced by the pre-warmed StemPro Adipogenesis medium (22.25 ml StemPro® Adipocyte Differentiation Basal Medium (Gibco)+2.5 ml StemPro Adipogenesis supplement (Gibco)+250 μl PS) and the incubation is continued in the CO₂ incubator. MSCs expand as they differentiate in the adipogenic conditions. Medium is replaced with fresh pre-warmed Adipogenesis medium every 3-4 days. On day 21 or later, adipogenesis can be analyzed using the Oil Red O stain analysis.

### Oil Red O Stain Analysis

The medium is removed from the wells of the 12-well plate. Cells are rinsed once with PBS (1 ml). PBS is removed from the wells and cells are fixed with 4% formaldehyde solution (diluted in PBS) for 30 minutes. After fixation, the cells are rinsed twice with distilled water and stained with Oil Red O solution (6 ml Oil Red O stock solution (Merck)+4 ml deionized water) for 20 minutes on a plate shaker at room temperature. The cells are then rinsed 3-4 times with 2 ml deionized water, visualized under the light microscope and images are taken.

### Example 6.5.3: Chondrogenic differentiation

The iPSC-derived MSCs are seeded at 200.000 cells in 15 ml tubes with 1 ml StemMACS ChondroDiff Medium (Miltenyi Biotec), optionally including PS. The tube is placed on a stand with the lid placed on it without tightening and incubated overnight in a CO₂ incubator. Duplicates or triplicates are maintained. As a negative control, 200.000 cells are seeded in 15 ml tubes with 1 ml of the MSCG medium. MSC spheroids are formed spontaneously within 24 to 48 hours. Medium is carefully replaced by fresh pre-warmed StemMACS ChondroDiff Medium, optionally including PS, every 3 to 4 days. Approximately 50-100 μl medium is kept left, in order to avoid aspirating the spheroids. It is ensured that the spheroids do not stick to the bottom of the tube. Incubation is performded for 21 days. On day 21 or later, chondrogenesis can be analyzed using the Alcian blue stain analysis.

### Alcian Blue Stain Analysis

After the differentiation time is completed, the medium is carefully removed. The spheroids are gently washed with PBS (1 ml). PBS is then removed from the tubes. The spheroids are fixed with 4% formaldehyde solution (diluted in PBS) for 3 hours. After fixation, the spheroids are transferred to plates. This makes it easier to handle the spheroids. The fixative is aspirated, the spheroids are washed twice with distilled water and stained with Alcian blue staining solution, followed by incubation in the dark for 45 minutes. Alcian blue staining solution is carefully removed with a 1 ml pipette, retaining the spheroids. The spheroids are then washed twice with deionized water for 10 minutes each. PBS is added, the cartilage spheroids are analyzed and images are taken. The cartilage spheroids stain dark blue, the negative control stains light blue.

### Example 7

### Chromatin Immunoprecipitation

Chromatin immunoprecipitation experiments are performed using the Magnetic Low cell ChIP Kit from Diagenode following the manufacturer's instructions and using 15,000 cells per immunoprecipitation. Antibodies used are from Millipore 07-440 (antiH3K27me3), 07-030 (anti-H3K4me2) and 17-625 (anti-H3K9me3).

### Example 8

### Promoter Methylation Analysis

Genomic DNA is extracted by samples of about 500.000 CD 133+ and CBiPS cells using QIA AMP DNA Mini Kit (Qiagen). Two micrograms of purified DNA is mutagenized with Epitect Bisulfite Kit (Qiagen) according to manufacturer specifications. The promoter sequences of interest are amplified by two subsequent PCRs using primers previously described. The resulting amplified products are cloned into pGEM T Easy plasmids, amplified in TOP cells, purified and sequenced.

### Example 9: Extra Cellular Vesicles (EV) Preparation

### Example 9.1:

Extra cellular vesicles (EV) may be prepared as follows; See Madel et al “Promoter methylation analysis ( . . . ), Bio RX (2020). For EV harvesting, conditioned media (CMs) is thawed and further purified following 45 min 6,800×g centrifugation (Rotor: JS-5.3) by a subsequent 0.22 μm filtration step using rapid flow filter (Nalgene, Thermo Fisher Scientific). EVs are precipitated in 10% polyethylene glycol 6000 (PEG) and 75 mM sodium chloride (NaCl) by overnight incubation and subsequent centrifugation at 1,500×g and 4° C. for 30 min as described previously (Kordelas et al., 2014; Ludwig et al., 2018). Pelleted EVs are re-suspended and washed with sterile 0.9% NaCl solution (Braun, Nelsungen, Germany) to remove contaminating soluble proteins. Next, EVs are re-precipitated by ultracentrifugation at 110,000×g for 130 min (XPN-80, Ti45 rotor, kfactor: 133). Finally, EV pellets are re-suspended in 10 mM HEPES 0.9% NaCl buffer (Thermo Fisher Scientific). Concentration is adjusted so that 1 mL final sample contained the EV yield prepared from CM of approximately 4×10⁷ MSC equivalents. MSC-EV preparations are stored at −80° C. Repetitive thawing and freezing cycles are avoided. For control purposes, fresh platelet media (PL) is processed in parallel (including incubation for 48 hours at 37° C., 5% CO₂, saturated water vapor atmosphere). The following article is helpful for establishing the best protocol; See Scaled Isolation of Mesenchymal Stem/Stromal Cell-Derived Extracellular Vesicles Verena Börger, Simon Staubach, Robin Dittrich, Oumaima Stambouli, Bernd Giebel-First published: 21 Sep. 2020 https://doi.org/10.1002/cpsc.128. The method described is applicable for the large-scale isolation of EVs from CM of various cell types. EVs harvested with this method from MSC-conditioned media have been successfully applied to various preclinical models. EVs share several features with viruses, such as their size and a comparable molecular assembly. Since viruses can be concentrated by PEG precipitation, the authors adopted, and optimized protocols originally developed for viruses to be used with EVs. The principle of the PEG precipitation is based on replacement of water molecules that form a hydrate envelope around the EVs. Due to the hydrophobic effect, the EVs precipitate surrounded by PEG, leading to a massive volume reduction, mandatory for using subsequent ultracentrifugation-based methods. The authors established the method as a low-cost alternative to commercially available EV precipitation products. As an alternative method, TFF is preferred. Depending on the system, TFF can be scaled to process hundreds of liters in relatively short time intervals. TFF devices from some companies are provided as automated, scalable systems, both as benchtop devices for research labs and as big machines for industry.

Although the described method can be scaled for EV preparation, there are some bottlenecks to be discussed. The method needs to be considered an open system, including numerous handling steps that increase the risk of contamination. The method is only scalable to mid-range. The limiting factor is the centrifuge size. For example, only up to 5 L can be processed if only one ultracentrifuge run is going to be performed to remove residual PEG from the samples. In total, this is still 14-fold more than the amount that can be processed by conventional differential centrifugation protocols. For larger volumes, several runs need to be performed.

### Example 9.2

Most preferably, EVs are prepared as follows: Starting at 50% confluence of the MSCs, the medium is exchanged and the conditioned media (CM) are collected after 48 h, until cells reach 80-90% confluency. In total, 200 ml of CM (two Nunc Triple Flasks) are collected from each passage (e.g. p11, p13, . . . , p19). For clearance before storing at −20° C., the CM are pre-processed by 15 min centrifugation at 2.000×g and 4° C. Supernatants are transferred into new containers and stored at −20° C. until further processing. For preparation of the EVs, frozen CM are thawed and EVs are prepared via polyethylene glycol precipitation followed by ultracentrifugation (PEG-UC) exactly as described previously [Ludwig et al., Precipitation with polyethylene glycol followed by washing and pelleting by ultracentrifugation enriches extracellular vesicles from tissue culture supernatants in small and large scales. J Extracell Vesicles. 2018;7(1):1528109. Epub 2018/10/26. doi: 10.1080/20013078.2018.1528109. PubMed d PMID: 30357008; PubMed Central PMCID: PMCPMC6197019 and Kordelas et al., MSC-derived exosomes: a novel tool to treat therapy-refractory graft-versus-host disease. Leukemia. 2014;28(4):970-3. Epub 2014/01/22. doi: 10.1038/leu.2014.41. PubMed PMID: 24445866]. Typically, the EV yield of CM 4×10⁷ cells are dissolved in 1 ml. For example, the CM from e.g. p17-19 can be pooled (3 times 200 ml) and the EVs can be prepared. Aliquots of the obtained EV sample are characterized by imaging flow cytometry (IFCM) for the three classical EV markers CD9, CD63 and CD81. For each marker, EVs obtained from CM of 100.000 cell equivalents (2.5 μl) are labelled by methods known in the art and analyzed. Labelled EVs are analyzed by IFCM.

### Example 10: Mixed Lymphocyte Reaction Assay

### Example 10.1

To test allogeneic immune responses, a novel multi-donor mixed lymphocyte reaction assay (MLR) is used (Bremer et al.). Briefly, peripheral blood mononuclear cells (PBMC) of 12 donors are harvested from buffy coats via conventional Ficoll density gradient centrifugation (Beckmann et al., 2007; Giebel et al., 2004), pooled, and stored in the vapor phase of liquid nitrogen. Upon thawing, PBMCs are cultured in RPMI 1640 medium (Thermo Fisher Scientific) supplemented with 10% human AB serum (produced in house) and 100 U/ml penicillin and 100 μg/ml streptomycin (Thermo Fisher Scientific). Mixed PBMCs are plated at densities of 600,000 cells per 200 μL and per well of 96-well u-bottom shape plates (Corning, Kaiserslautern, Germany) and cultured either in the presence or absence of MSC-EV preparations to be tested at 37° C. in a 5% CO₂ atmosphere. After 5days, cells are harvested, stained with a collection of specifically selected fluorescent labelled antibodies (CD4-BV785 [300554, Clone: RPA-T4, BioLegend]), CD25-PE [12-0259-42, Clone: BC-96, Thermo Fischer Scientific] and CD54-AF700 [A7-429-T100, Clone: 1H4, EXBIO]) and analysed on a Cytoflex flow cytometer (Software Cytexpert 2.3, Beckman-Coulter). Activated and non-activated CD4+ and/or CD8+ T cells are discriminated by means of their CD25 and CD54 expression, respectively. 25 μg of MSC-EV preparations to be tested are applied into respective wells.

### Example 10.2

Alternatively, the immunomodulatory potential of all obtained MSC-EV preparations are compared in a multi-donor mixed lymphocyte reaction (mdMLR) assay as described previously [Tertel et al., Imaging flow cytometry challenges the usefulness of classically used extracellular vesicle labeling dyes and qualifies the novel dye Exoria for the labeling of mesenchymal stromal cell-extracellular vesicle preparations. Cytotherapy. 2022;24(6):619-28. doi: https://doi.org/10.1016/j.jcyt.2022.02.003]. EV equivalents of 200,000 cells are used for this assay. Due to the mutual allogeneic stimulation within the PBMC mixture, reproducibly high portions of CD4⁺-and CD8⁺-T cells get activated. However, applying EVs with immunomodulatory potential leads to a significant reduction of activated T cells. Activated and non-activated CD4+ and/or CD8+ T cells are discriminated by either expressing CD25 and CD54 (activated) or not/very weakly expressing CD25 and CD54 (non-activated).

| | | | |
|---|---|---|---|
| Antibodies used | α-CD25 antibody | BD | CD25-PE-Cy5.5 |
| for detection of | | Biosciences | |
| activated T cells | α-CD54 antibody | EXBIO Praha | CD54-AF700 |

Alternative methods for preparing EVs from MSC-conditioned cell culture media, characterizing the EV preparations, labelling EVs, or IFCM analyses are described in Tertel et al. and are incorporated herein by reference (https://doi.org/10.1016/j.jcyt.2022.02.003).

### FIGURE CAPTIONS

FIG. 1: Mesenchymal stromal/stem cells (MSCs) differentiated from human induced pluripotent stem cells (hiPSCs) using WNT activation exhibit a fibroblastoid morphology and adhere to Laminin-coated plates/wells, as well as to plastic. Differentiation was achieved by seeding hiPSC R26-6 on 6-wells pre-coated with Laminin 1 hour earlier. The next day, cells were treated with 4 μM of the WNT activator CHIR99021 in XF medium supplemented with penicillin-streptomycin (PS). Treatment lasted for six days and cells were subsequently cultured in low-glucose MSC medium with 10% FCS. P1=first passage after WNT-induced differentiation of hiPSCs to MSCs. E1AP1=passage 1 (P1) hiPSC-derived MSCs. E1BP1=passage 1 (P1) hiPSC-derived MSCs, which were treated with a WNT inhibitor (C59) for the first 4 days in low-glucose MSC medium with 10% FCS.

FIG. 2: Mesenchymal stromal/stem cells (MSCs) differentiated from human induced pluripotent stem cells (hiPSCs) using WNT activation exhibit a fibroblastoid morphology and adhere to plastic during several rounds of passaging. The cells were generated as described for FIG. 1. Different passages after WNT-induced differentiation of hiPSCs to MSCs (P1-P5) are shown.

FIG. 3: Cell density does not affect hiPSC differentiation towards MSCs. The hiPSC line R26-6 was seeded at different cell densities (2.5K, 5K, 10K, 20K, 40K cells/cm²) in 6 well-plates with XF medium+PS). The following day, the cells were treated with XF medium+PS+4 μM CHIR99021 (CH) for 6 days. After 6 days, the medium was replaced with low glucose MSC medium+10% FCS. Cells were cultured in MSC medium+10% hPL from passage 2 onwards.

FIG. 4: Growth kinetics of the MSCs derived from different RCT hiPSC lines and with differing culturing conditions after induction of differentiation.
(A) Cumulative growth curve of hiPSC (R26-6)-derived MSCs. Error bars indicate standard deviation from three independent experiments, wherein either 10% FCS-(and for one experiment also 10% hPL-comprising medium from P2 onwards)-comprising medium was used from day 6 after induction of differentiation onwards.
(B) Cumulative growth curve of hiPSC (R26-6)-derived MSCs, which were cultured under serum-free, defined media conditions from day 6 after induction of differentiation onwards.
(C) Cumulative growth bar plots of MSCs, which were derived from two different hiPSC lines (R23 and R25) and cultured in low glucose MSC medium+10% hPL from day 6 after induction of differentiation onwards.

FIG. 5: MSCs derived from different hiPSC lines (R23, R25 and R26-6) all express the key MSC markers CD44, CD73 and CD90 and lowly express CD105. MSCs were cultured in low glucose MSC medium+10% hPL from day 6 after induction of differentiation onwards. IgG was used as a control and indicates background fluorescence.

FIG. 6: MSCs derived from different hiPSC lines (R23, R25 and R26-6) all do not express the non-MSC, other lineage markers. MSCs were cultured in low glucose MSC medium+10% hPL from day 6 after induction of differentiation onwards. IgG was used as a control and indicates background fluorescence. The other lineage markers refer to: endothelial (CD31) and hematopoietic (CD31, CD34, CD45), macrophages (CD14, HLA-DR), lymphocytes and monocytes (HLA-DR), and induced pluripotent stem cells (TRA 1-60).

FIG. 7: The key MSC markers (CD44, CD73 and CD90) are consistently and highly expressed from early to late MSC passages, but expression of CD105 is variable. hiPSC (R26-6)-derived MSCs were cultured in low glucose MSC medium+10% hPL from day 6 after induction of differentiation onwards. 100% or almost 100% of cells are positive for CD90 and CD44 throughout the passages. Always at least 85% of cells express CD73. Expression of CD105 is more variable.

FIG. 8: hiPSC (R26-6)-derived MSCs cultivated in serum-free, defined medium express the key MSC markers CD44, CD73, CD90 and CD105. (Left panels) MSCs were either cultured in StemMacs MSC Expansion Media XF supplemented with StemMacs MSC Expansion Media XF Supplement (Miltenyi Biotec; 700 μl/50 ml) and 1:100 Penicillin-Streptomycin (PS; Thermo), or (right panels) MSCs were cultured in Sartorius medium, with both of the options starting from day 6 after induction of differentiation onwards. IgG was used as a control and indicates background fluorescence.

FIG. 9: hiPSC (R26-6)-derived MSCs cultivated in serum-free, defined medium do not express the non-MSC, other lineage markers. MSCs were cultured in StemMacs MSC Expansion Media XF supplemented with StemMacs MSC Expansion Media XF Supplement (Miltenyi Biotec; 700 μl/50 ml) and 1:100 Penicillin-Streptomycin (PS; Thermo) from day 6 after induction of differentiation onwards. IgG was used as a control and indicates background fluorescence.

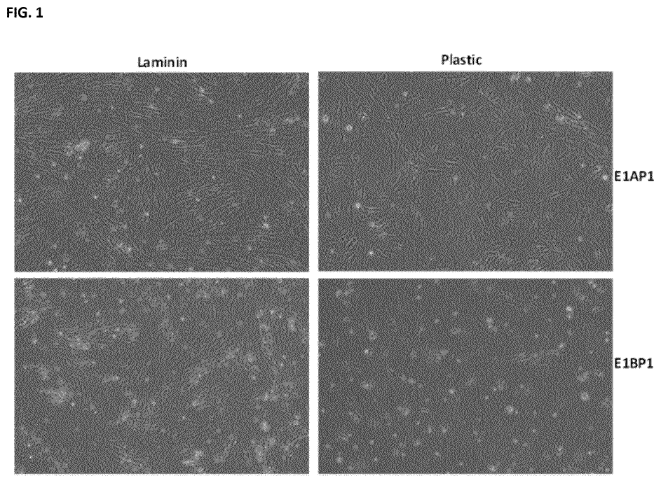

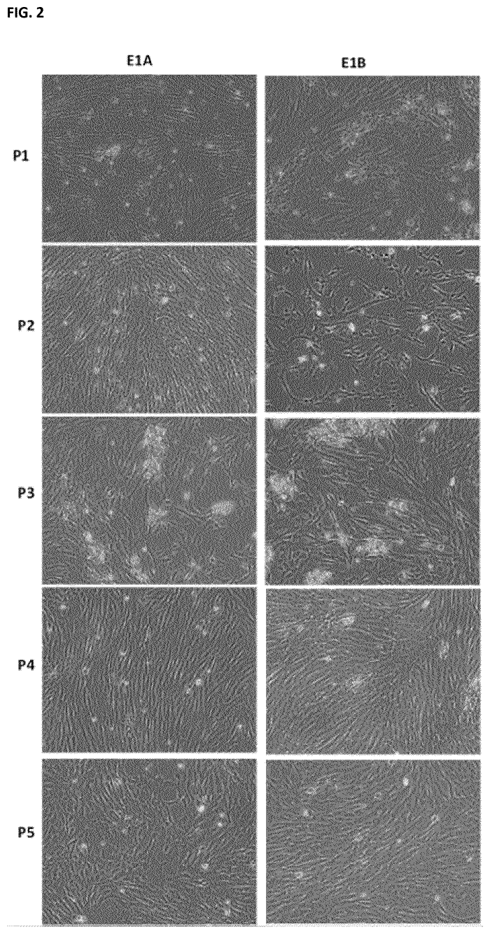

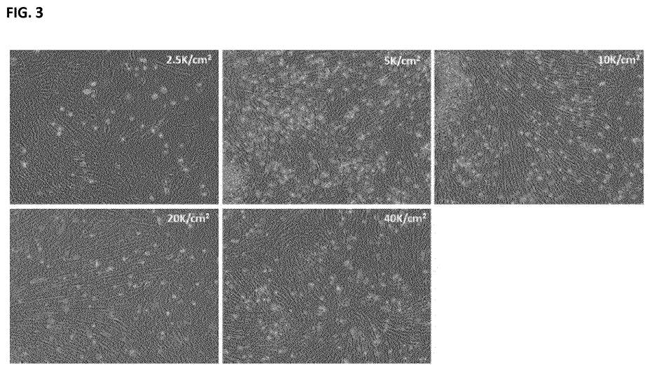

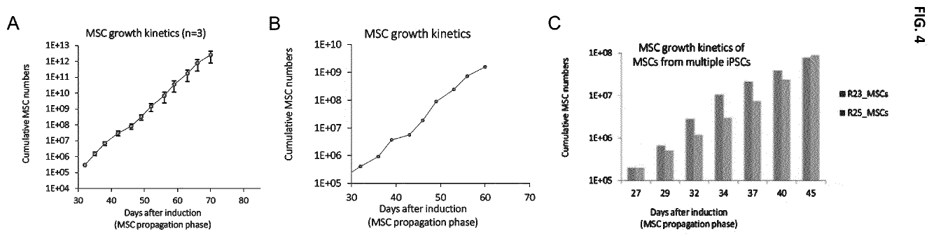

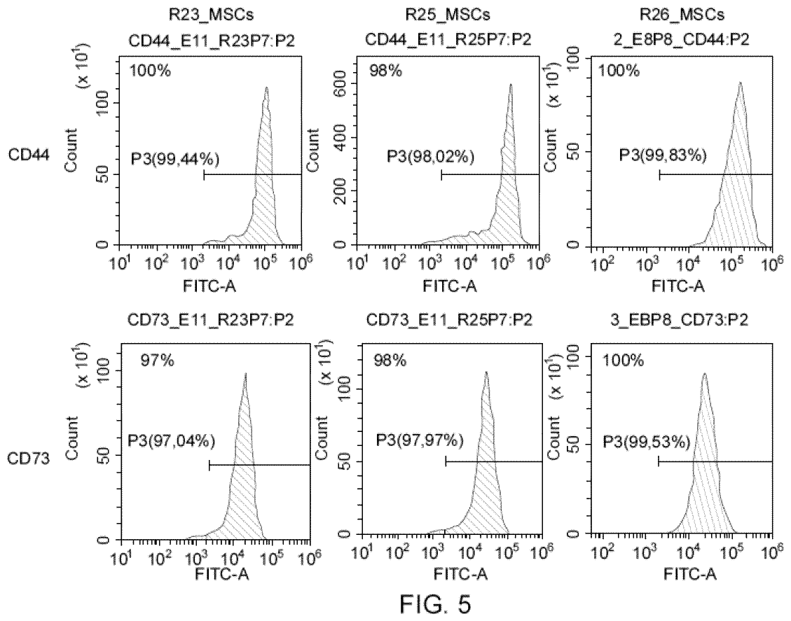

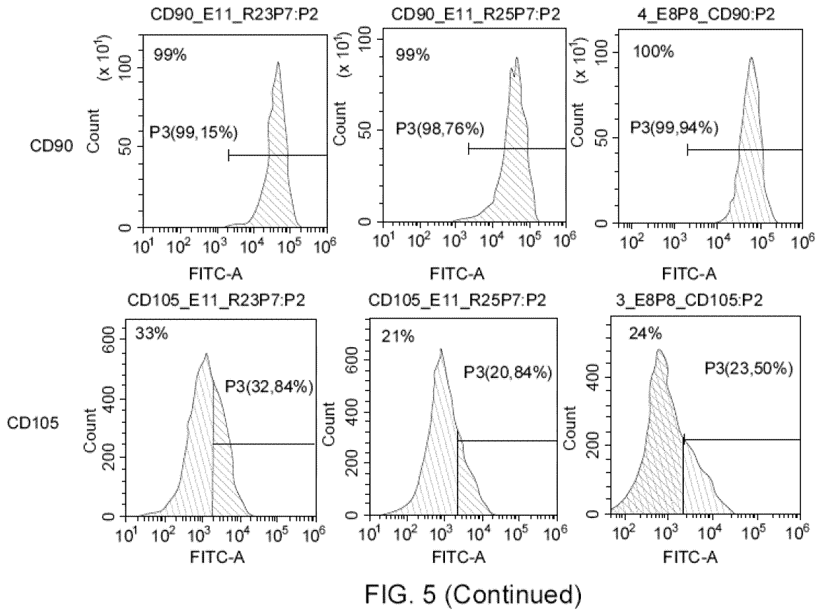

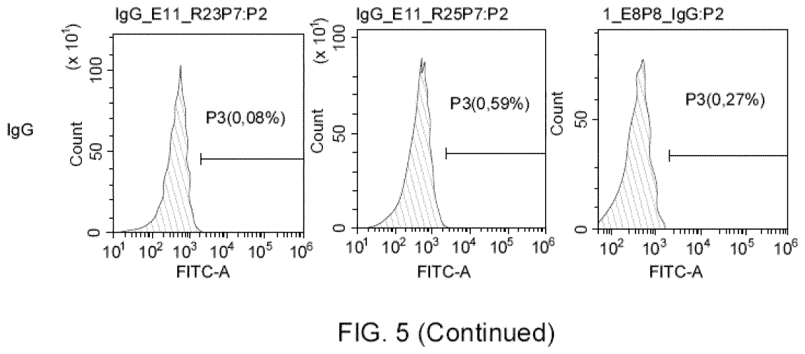

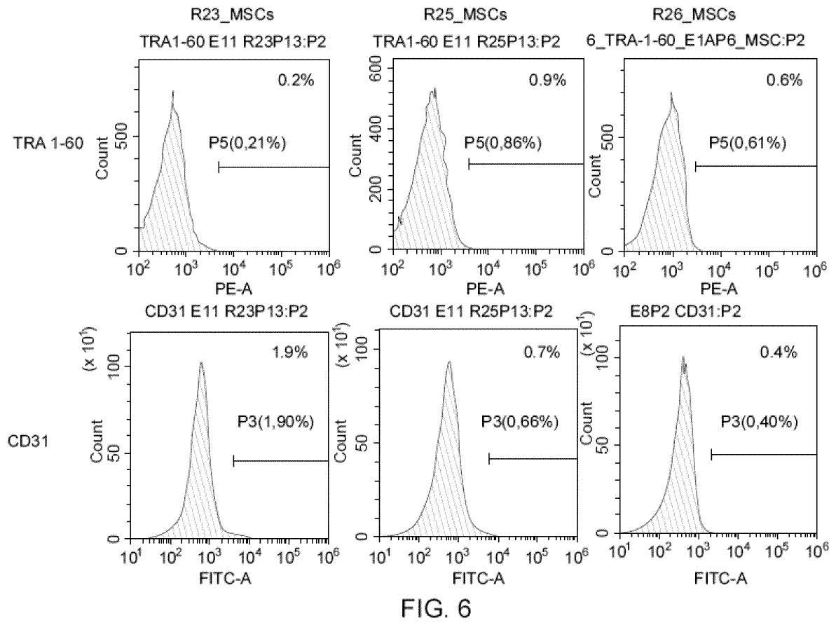

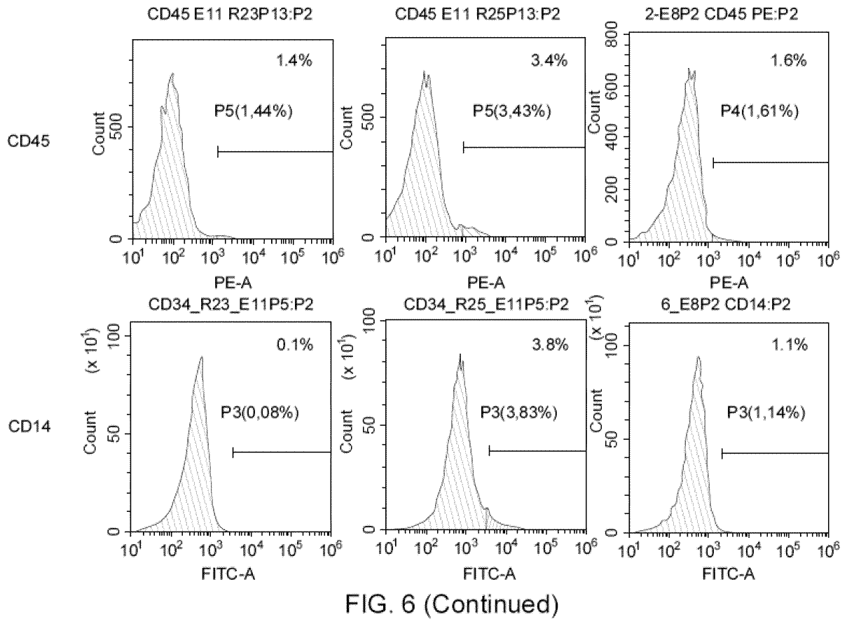

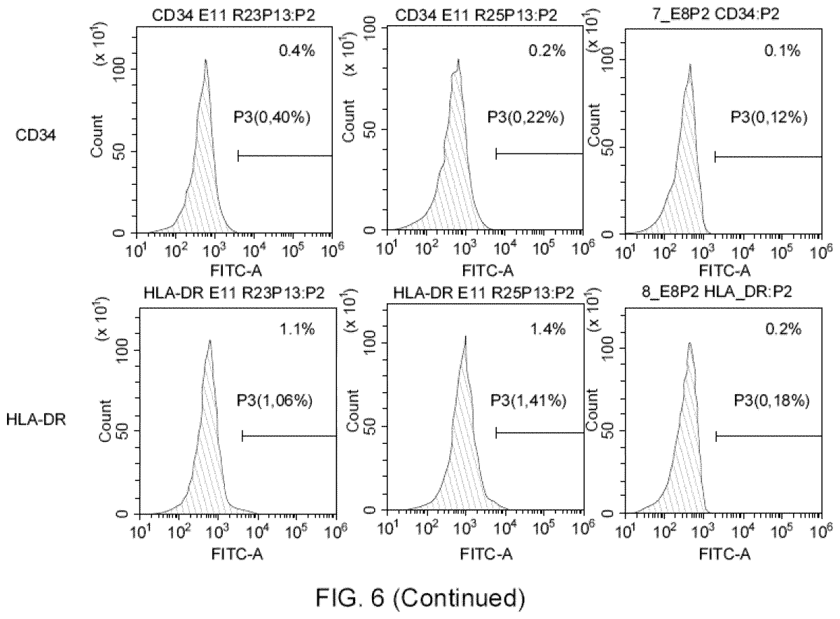

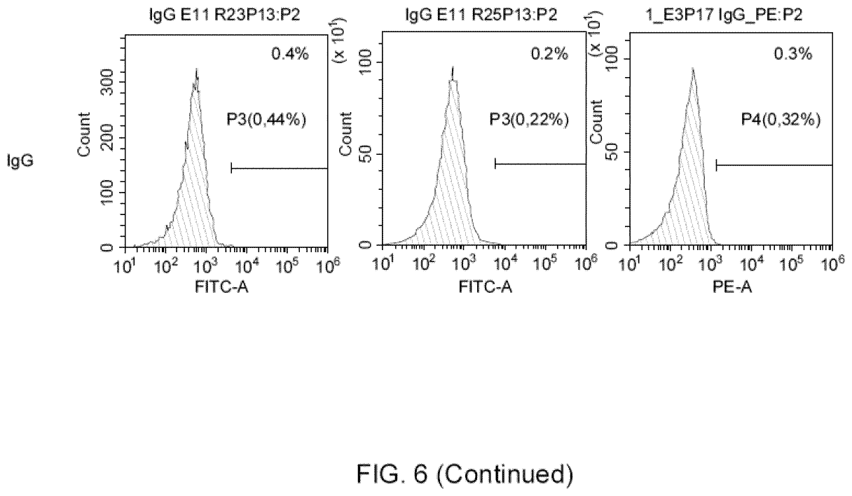

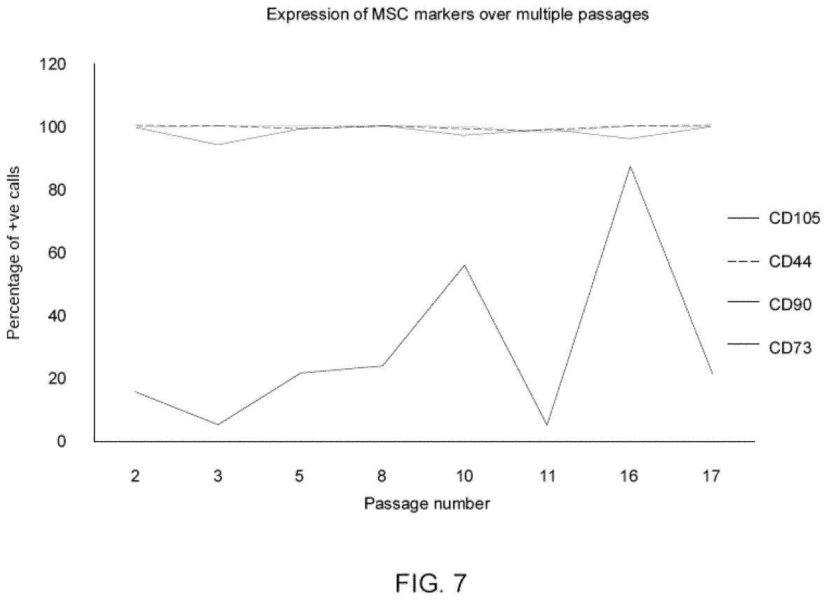

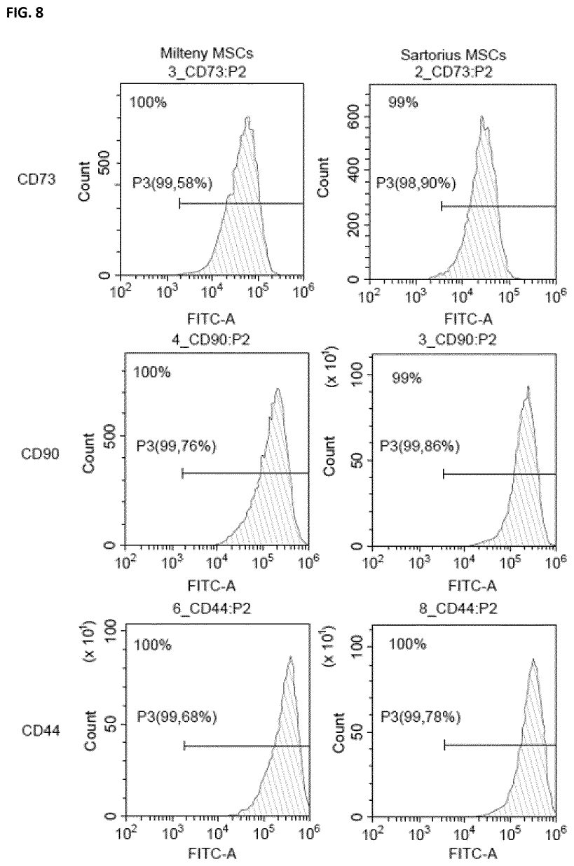

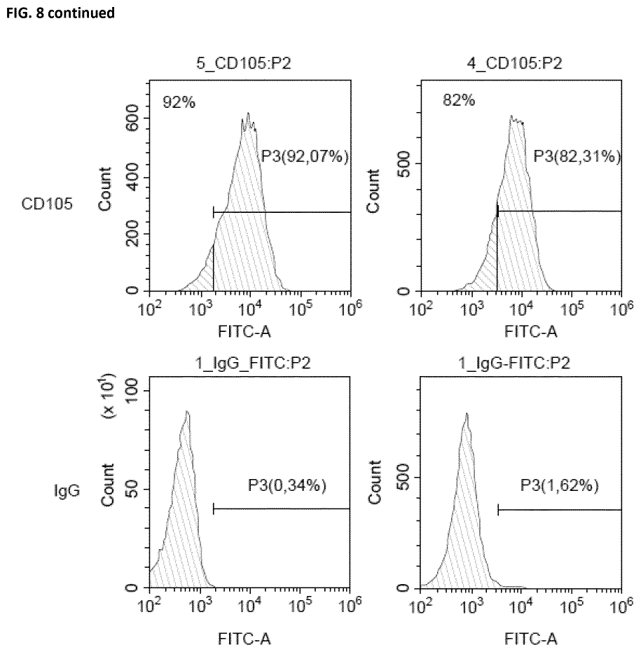

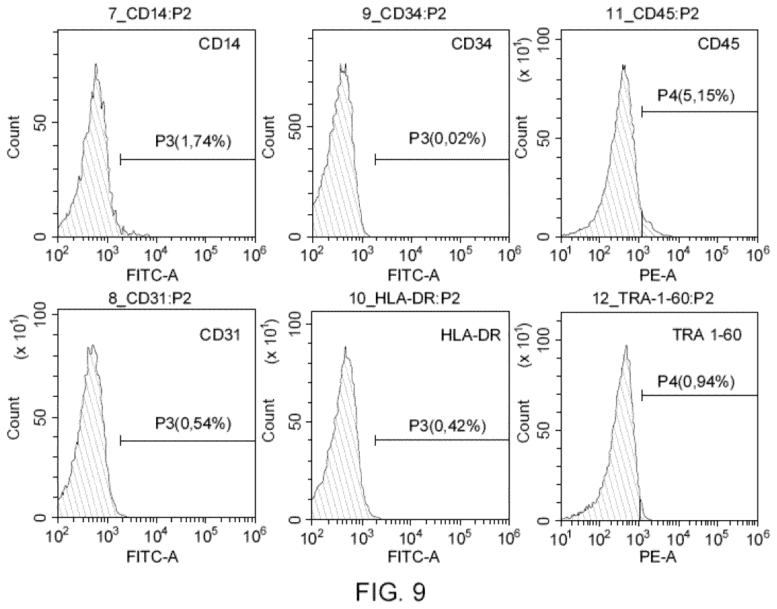

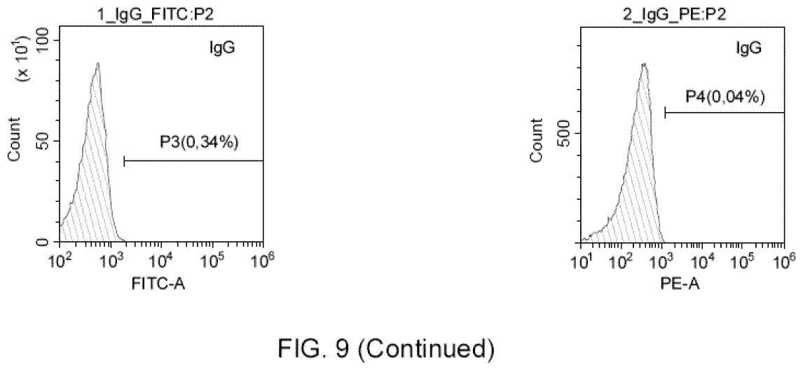

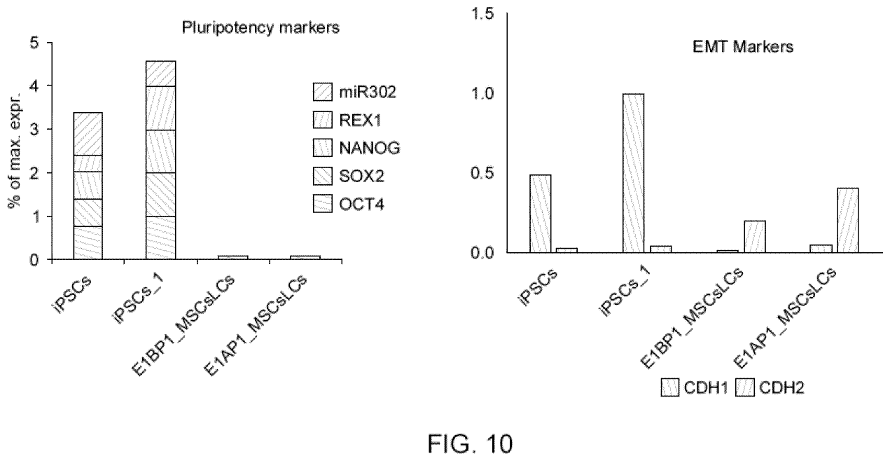

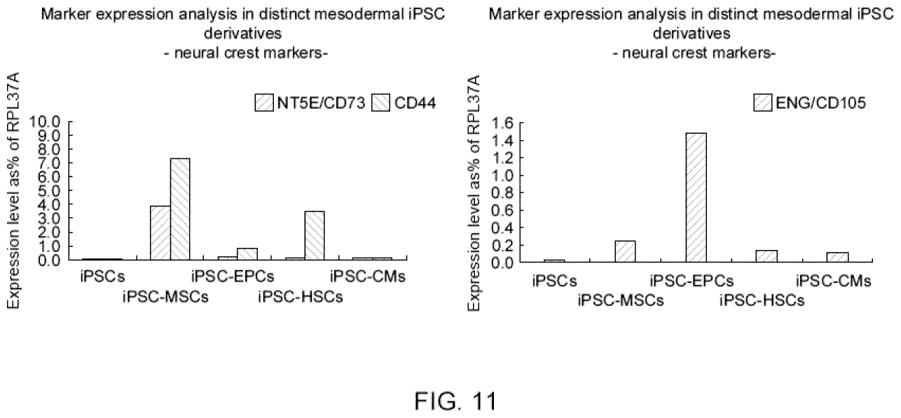

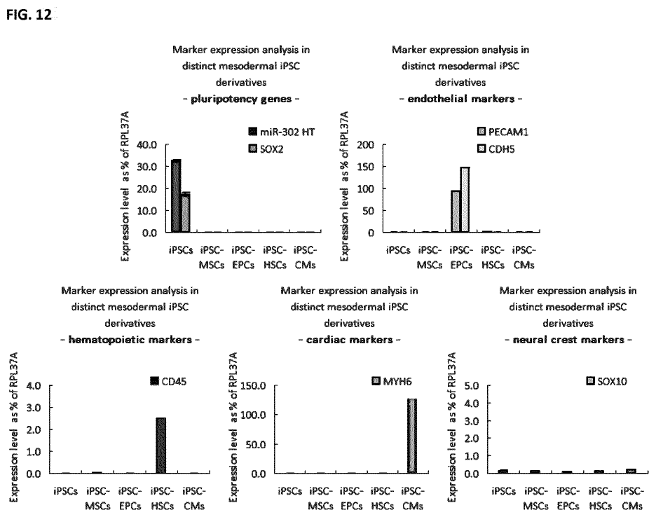

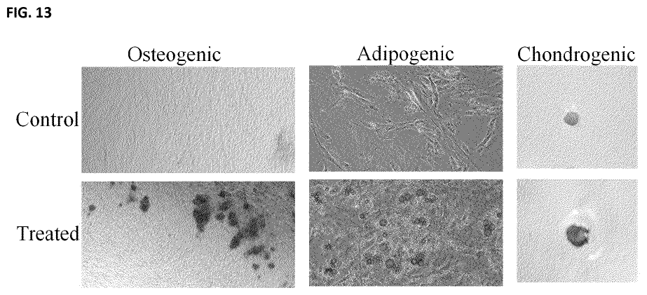

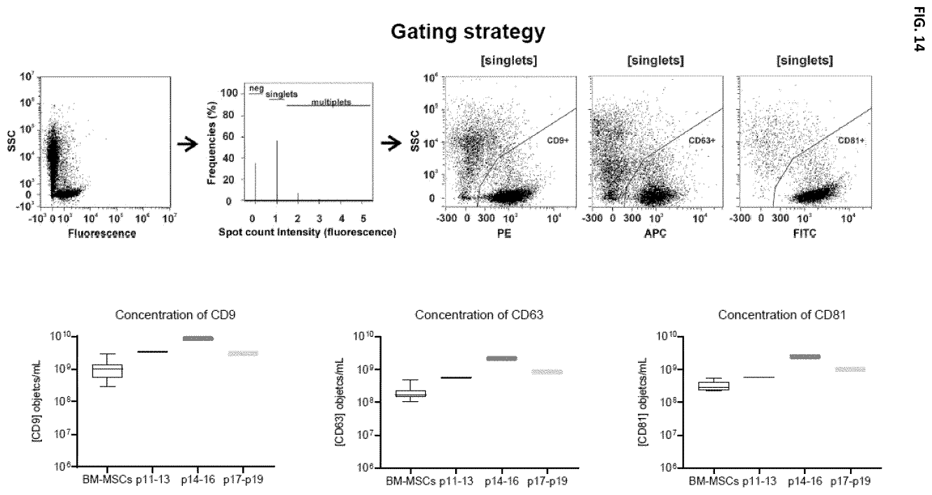

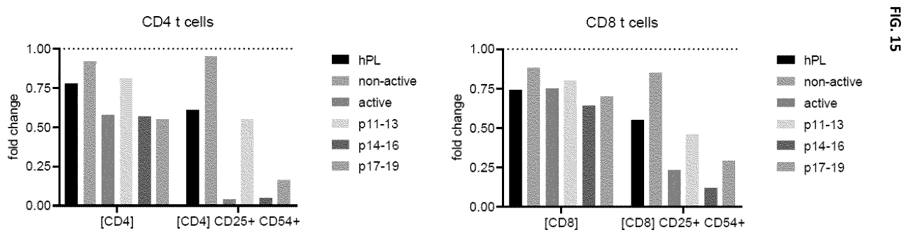

FIG. 10: hiPSC (R26-6)-derived MSCs do not express (mRNA) pluripotency markers (left panel) and the epithelial marker CDH1 (right panel; blue bars), but do express the mesenchymal marker CDH2 (right panel; orange bars). Starting from day 6 after induction of differentiation, the cells were cultured in low-glucose MSC medium with 10% FCS (MSCs are the same as the ones depicted in FIG. 1).

FIG. 11: qRT-PCR of the MSC markers in iPSCs (n=2; line R26), iPSC-derived MSCs (iPSC-MSCs) (n=3: P7, P11, and P11, respectively) and other mesodermal cell types derived from iPSCs: Endothelial Progenitor Cells (iPSC-EPCs), Hematopoietic Stem Cells (iPSC-HSCs) and Cardiomyocytes (iPSC-CMs). Mean expression levels are shown as % of RPL37A expression, which was used as a housekeeping gene.

FIG. 12: qRT-PCR of non-MSC/other mesodermal markers in iPSCs (n=2; line R26), iPSC-derived MSCs (iPSC-MSCs) (n=3: P7, P11, and P11, respectively) and other mesodermal cell types derived from iPSCs: Endothelial Progenitor Cells (iPSC-EPCs), Hematopoietic Stem Cells (iPSC-HSCs) and Cardiomyocytes (iPSC-CMs). Mean expression levels are shown as % of RPL37A expression, which was used as a housekeeping gene.

FIG. 13: hiPSC (R26-6)-derived MSCs differentiated into osteoblasts (after Alizarin Red S staining), adipocytes (Oil Red O staining), and chondrocytes (Alcian Blue staining). Starting from day 6 after induction of differentiation, the cells were cultured in StemMacs MSC Expansion Media XF supplemented with StemMacs MSC Expansion Media XF Supplement (Miltenyi Biotec; 700 μl/50 ml) and 1:100 Penicillin-Streptomycin (PS; Thermo). Osteogenic/adipogenic/chondrogenic differentiation of MSCs was then performed as described in Example section 6.5.

FIG. 14: Imaging flow cytometry (IFCM) analysis of MSC-derived extracellular vesicles (EVs). EVs were prepared as described in Example section 9.2. Aliquots of MSC-EV preparations (p11-p13, p14-p16 and p17-p19) from MSCs, which were cultured in low glucose MSC medium with 10%hPL, were analyzed for the presence of CD9+, CD63+ and CD81+ EVs by IFCM. Aliquots of a representative bone marrow (BM)-MSC-EV sample were used as controls.

FIG. 15: Immunomodulatory potential of MSC-EVs. A multi-donor mixed lymphocyte reaction (mdMLR) assay (see also example 10.2) was performed on aliquots of MSC-EV preparations (p11-p13, p14-p16 and p17-p19) from MSCs, which were cultured in low glucose MSC medium with 10% hPL. The immunomodulatory abilities of these MSC-EV preparations were investigated in comparison to the controls, i.e. in the presence of active/non-active BM-MSC-EV samples or EVs prepared from fresh, hPL-supplemented culture media. The content of CD4⁺ and CD8⁺ T cells were analyzed for expression of the activation markers CD25 and CD54. Results are depicted as ratios of the content of activated T cells of EV-treated mdMLRs to that of EV-untreated mdMLRs (fold change).

1-14. (canceled)

15. A method of producing mesenchymal stem cell derived exosomes comprising the steps of,
i) performing a method of producing a clonal mesenchymal stem cell line capable of producing exosomes (MSC-derived extracellular vesicles) comprising the steps of,
a. providing human induced pluripotent stem cells (hiPSCs),
b. generating therefrom an immortalized clonal cell line of mesenchymal stem cells (IMSCs),
c. characterizing the potential of the IMSCs to produce exosomes,
ii) cultivating the desired IMSCs such that the IMSCs produce exosomes,
iii) isolating the exosomes.

16. The method according to claim 15, wherein the exosome have a density as determined by density gradient centrifugation of 1.13 g per mL to 1.19 g per mL.

17. The method according to claim 15, wherein the hiPSCs have a homozygous leukocyte antigen (HLA) haplotype (HLA-homo HP).

18. The method according to claim 15, wherein the hiPSCs were generated by means of one or more Yamanaka factors such as but not limited to Myc, Oct3, Oct4, Sox2 and Klf4 and stem from preferably umbilical cord blood tissue.

19. The method according to claim 15, wherein the method encompasses transfecting an umbilical cord blood stem cell with a nucleic acid encoding an OCT4 protein and a nucleic acid encoding a SOX2 protein to form a transfected cord blood stem cell and allowing said transfected cord blood stem cell to divide thereby forming said induced pluripotent stem cell.

20. The method according to claim 15, wherein generation of immortalized clonal cell line of mesenchymal stem cells (IMSC) is done by means of a tissue specific media, such as a cardiomyogenic medium (CARM) and optionally a specific p38-MAPK inhibitor, wherein after generation the resulting IMSC expresses one or more of the following markers selected from the group of CD29, CD44, CD73, CD90, and CD105.

21. The method according to claim 15, wherein characterization is done by a method selected from the group of, i) performing a potency test for the exosomes produced, ii) testing the size distribution of the exosomes produced and iii) testing for a exosomal surface marker, wherein preferably the exosomal surface marker is selected from the group of CD 63, CD 9, Calnexin, Hsp70 and TSG101.

22. The method according to claim 15, wherein the immortalized clonal cell line of mesenchymal stem cells (IMSC) are characterized by their capability to form fat or bone cells.

23. An EV produced according to the method of claim 15.

24. An EV produced according to the method of claim 17.

25. An EV produced according to the method of claim 19.

26. A method of treating a patient in need thereof, with an EV according to claim 23.

27. A method of treating a patient in need thereof, with an EV according to claim 24.

28. A method of treating a patient in need thereof, with an EV according to claim 25.

29. A method of treating a patient in need thereof, with an EV according to claim 23, wherein the disease selected from the group of bone marrow, skin, heart, and corneal transplantation, graft versus host disease, hepatic and renal failure, lung injury, bronchopulmonary dysplasia, rheumatoid arthritis, treatment of autoimmune diseases such as Crohn's disease, ulcerative colitis, multiple sclerosis, lupus and diabetes; prevention of allograft rejection, neurological disorders and cardiovascular medicine; as well as Acute lymphoblastic leukemia (ALL), Acute myeloid leukemia (AML), Burkitt's lymphoma, Chronic myeloid leukemia (CML), Juvenile myelomonocytic leukemia (JMML), Non-Hodgkin's lymphoma Hodgkin's lymphoma, Lymphomatoid granulomatosis, Myelodysplastic syndrome (MDS), Chronic myelomonocytic leukemia (CMML), Bone Marrow Failure Syndromes, Amegakaryocytic thrombocytopenia, Autoimmune neutropenia (severe), Congenital dyserythropoietic anemia, Cyclic neutropenia, Diamond-Blackfan anemia, Evan's syndrome, Fanconi anemia, Glanzmann's disease, Juvenile dermatomyositis, Kostmann's syndrome, Red cell aplasia, Schwachman syndrome, Severe aplastic anemia, Congenital sideroblastic anemia, Thrombocytopenia with absent radius (TAR syndrome), Dyskeratosis congenital, Blood Disorders, Sickle-cell anemia (hemoglobin SS), HbSC disease, Sickle βo Thalassemia, α-thalassemia major (hydrops fetalis), β-thalassemia major (Cooley's anemia), β-thalassemia intermedia, E-βo thalassemia, E-β+ thalassemia, Metabolic Disorders, Adrenoleukodystrophy Gaucher's disease (infantile), Metachromatic leukodystrophy, Krabbe disease (globoid cell leukodystrophy), Gunther disease, Hermansky-Pudlak syndrome, Hurler syndrome, Hurler-Scheie syndrome, Hunter syndrome, Sanfilippo syndrome, Maroteaux-Lamy syndrome, Mucolipidosis Type II, III, Alpha mannosidosis, Niemann Pick Syndrome, type A and B, Sandhoff Syndrome, Acute liver failure, Tay-Sachs Disease, Batten disease (inherited neuronal ceroid lipofuscinosis), Lesch-Nyhan disease, Immunodeficiencies, Ataxia telangiectasia, Chronic granulomatous disease, DiGeorge syndrome, IKK gamma deficiency, Immune dysregulation polyendocrineopathy, X-linked Mucolipidosis, Type II, Myelokathexis X-linked immunodeficiency, Severe combined immunodeficiency, Adenosine deaminase deficiency, Wiskott-Aldrich syndrome, X-linked agammaglobulinemia, X-linked lymphoproliferative disease, Omenn's syndrome, Reticular dysplasia, Thymic dysplasia, Leukocyte adhesion deficiency, Other Osteopetrosis, Langerhans cell histiocytosis, Hemophagocytic lymphohistiocytosis, Acute & Chronic Kidney Disease, Acute Kidney failure, Alzheimer's disease, Anti-Aging, Arthritis, Asthma, Cardiac Stem Cell Therapy, Cerebral Infarction (Stroke), Cerebral Palsy (Stroke), Chronic Obstructive Pulmonary Disease (COPD), Congestive Heart Failure, Diabetes Mellitus (Type I & II), Fibromyalgia, Immune Deficiencies, Ischemic Heart Disease, Lupus, Multiple Sclerosis, Myocardial/Cardiac Infarction, Heart failure, Osteoarthritis, Osteoporosis, Parkinson's Disease, Peripheral Arterial Disease, Rheumatoid Arthritis, Stem Cell Therapy in Plastic Surgery, Traumatic Brain Injury and Neurological Diseases.

30. A method of treating a patient in need thereof, with an EV according to claim 24, wherein the disease selected from the group of bone marrow, skin, heart, and corneal transplantation, graft versus host disease, hepatic and renal failure, lung injury, bronchopulmonary dysplasia, rheumatoid arthritis, treatment of autoimmune diseases such as Crohn's disease, ulcerative colitis, multiple sclerosis, lupus and diabetes; prevention of allograft rejection, neurological disorders and cardiovascular medicine; as well as Acute lymphoblastic leukemia (ALL), Acute myeloid leukemia (AML), Burkitt's lymphoma, Chronic myeloid leukemia (CML), Juvenile myelomonocytic leukemia (JMML), Non-Hodgkin's lymphoma Hodgkin's lymphoma, Lymphomatoid granulomatosis, Myelodysplastic syndrome (MDS), Chronic myelomonocytic leukemia (CMML), Bone Marrow Failure Syndromes, Amegakaryocytic thrombocytopenia, Autoimmune neutropenia (severe), Congenital dyserythropoietic anemia, Cyclic neutropenia, Diamond-Blackfan anemia, Evan's syndrome, Fanconi anemia, Glanzmann's disease, Juvenile dermatomyositis, Kostmann's syndrome, Red cell aplasia, Schwachman syndrome, Severe aplastic anemia, Congenital sideroblastic anemia, Thrombocytopenia with absent radius (TAR syndrome), Dyskeratosis congenital, Blood Disorders, Sickle-cell anemia (hemoglobin SS), HbSC disease, Sickle βo Thalassemia, α-thalassemia major (hydrops fetalis), β-thalassemia major (Cooley's anemia), β-thalassemia intermedia, E-βo thalassemia, E-β+ thalassemia, Metabolic Disorders, Adrenoleukodystrophy Gaucher's disease (infantile), Metachromatic leukodystrophy, Krabbe disease (globoid cell leukodystrophy), Gunther disease, Hermansky-Pudlak syndrome, Hurler syndrome, Hurler-Scheie syndrome, Hunter syndrome, Sanfilippo syndrome, Maroteaux-Lamy syndrome, Mucolipidosis Type II, III, Alpha mannosidosis, Niemann Pick Syndrome, type A and B, Sandhoff Syndrome, Acute liver failure, Tay-Sachs Disease, Batten disease (inherited neuronal ceroid lipofuscinosis), Lesch-Nyhan disease, Immunodeficiencies, Ataxia telangiectasia, Chronic granulomatous disease, DiGeorge syndrome, IKK gamma deficiency, Immune dysregulation polyendocrineopathy, X-linked Mucolipidosis, Type II, Myelokathexis X-linked immunodeficiency, Severe combined immunodeficiency, Adenosine deaminase deficiency, Wiskott-Aldrich syndrome, X-linked agammaglobulinemia, X-linked lymphoproliferative disease, Omenn's syndrome, Reticular dysplasia, Thymic dysplasia, Leukocyte adhesion deficiency, Other Osteopetrosis, Langerhans cell histiocytosis, Hemophagocytic lymphohistiocytosis, Acute & Chronic Kidney Disease, Acute Kidney failure, Alzheimer's disease, Anti-Aging, Arthritis, Asthma, Cardiac Stem Cell Therapy, Cerebral Infarction (Stroke), Cerebral Palsy (Stroke), Chronic Obstructive Pulmonary Disease (COPD), Congestive Heart Failure, Diabetes Mellitus (Type I & II), Fibromyalgia, Immune Deficiencies, Ischemic Heart Disease, Lupus, Multiple Sclerosis, Myocardial/Cardiac Infarction, Heart failure, Osteoarthritis, Osteoporosis, Parkinson's Disease, Peripheral Arterial Disease, Rheumatoid Arthritis, Stem Cell Therapy in Plastic Surgery, Traumatic Brain Injury and Neurological Diseases.

31. A method of treating a patient in need thereof, with an EV according to claim 25, wherein the disease selected from the group of bone marrow, skin, heart, and corneal transplantation, graft versus host disease, hepatic and renal failure, lung injury, bronchopulmonary dysplasia, rheumatoid arthritis, treatment of autoimmune diseases such as Crohn's disease, ulcerative colitis, multiple sclerosis, lupus and diabetes; prevention of allograft rejection, neurological disorders and cardiovascular medicine; as well as Acute lymphoblastic leukemia (ALL), Acute myeloid leukemia (AML), Burkitt's lymphoma, Chronic myeloid leukemia (CML), Juvenile myelomonocytic leukemia (JMML), Non-Hodgkin's lymphoma Hodgkin's lymphoma, Lymphomatoid granulomatosis, Myelodysplastic syndrome (MDS), Chronic myelomonocytic leukemia (CMML), Bone Marrow Failure Syndromes, Amegakaryocytic thrombocytopenia, Autoimmune neutropenia (severe), Congenital dyserythropoietic anemia, Cyclic neutropenia, Diamond-Blackfan anemia, Evan's syndrome, Fanconi anemia, Glanzmann's disease, Juvenile dermatomyositis, Kostmann's syndrome, Red cell aplasia, Schwachman syndrome, Severe aplastic anemia, Congenital sideroblastic anemia, Thrombocytopenia with absent radius (TAR syndrome), Dyskeratosis congenital, Blood Disorders, Sickle-cell anemia (hemoglobin SS), HbSC disease, Sickle βo Thalassemia, α-thalassemia major (hydrops fetalis), β-thalassemia major (Cooley's anemia), β-thalassemia intermedia, E-βo thalassemia, E-β+ thalassemia, Metabolic Disorders, Adrenoleukodystrophy Gaucher's disease (infantile), Metachromatic leukodystrophy, Krabbe disease (globoid cell leukodystrophy), Gunther disease, Hermansky-Pudlak syndrome, Hurler syndrome, Hurler-Scheie syndrome, Hunter syndrome, Sanfilippo syndrome, Maroteaux-Lamy syndrome, Mucolipidosis Type II, III, Alpha mannosidosis, Niemann Pick Syndrome, type A and B, Sandhoff Syndrome, Acute liver failure, Tay-Sachs Disease, Batten disease (inherited neuronal ceroid lipofuscinosis), Lesch-Nyhan disease, Immunodeficiencies, Ataxia telangiectasia, Chronic granulomatous disease, DiGeorge syndrome, IKK gamma deficiency, Immune dysregulation polyendocrineopathy, X-linked Mucolipidosis, Type II, Myelokathexis X-linked immunodeficiency, Severe combined immunodeficiency, Adenosine deaminase deficiency, Wiskott-Aldrich syndrome, X-linked agammaglobulinemia, X-linked lymphoproliferative disease, Omenn's syndrome, Reticular dysplasia, Thymic dysplasia, Leukocyte adhesion deficiency, Other Osteopetrosis, Langerhans cell histiocytosis, Hemophagocytic lymphohistiocytosis, Acute & Chronic Kidney Disease, Acute Kidney failure, Alzheimer's disease, Anti-Aging, Arthritis, Asthma, Cardiac Stem Cell Therapy, Cerebral Infarction (Stroke), Cerebral Palsy (Stroke), Chronic Obstructive Pulmonary Disease (COPD), Congestive Heart Failure, Diabetes Mellitus (Type I & II), Fibromyalgia, Immune Deficiencies, Ischemic Heart Disease, Lupus, Multiple Sclerosis, Myocardial/Cardiac Infarction, Heart failure, Osteoarthritis, Osteoporosis, Parkinson's Disease, Peripheral Arterial Disease, Rheumatoid Arthritis, Stem Cell Therapy in Plastic Surgery, Traumatic Brain Injury and Neurological Diseases.

32. A pharmaceutical composition comprising an EV according to claim 23.

33. A pharmaceutical composition comprising an EV according to claim 24.

34. A pharmaceutical composition comprising an EV according to claim 25.
